(19) United States
(12) Patent Application Publication
(10) Pub. No.: US 2022/0125153 A1
(43) Pub. Date: Apr. 28, 2022

(54) VARIABLE MATERIAL PROPERTIES FOOT COVERING

(71) Applicant: Kambiz Behzadi, Pleasanton, CA (US)

(72) Inventor: Kambiz Behzadi, Pleasanton, CA (US)

(21) Appl. No.: 17/447,444

(22) Filed: Sep. 13, 2021

Related U.S. Application Data
(60) Provisional application No. 63/077,610, filed on Sep. 12, 2020.

(51) Int. Cl. A43B 7/00

(52) U.S. Cl. CPC A43B 7/00

(57) ABSTRACT

Structures and methods, as compared to conventional shoe cushioning, that may be employed to decrease, diagnose, and/or treat running injuries. A tessellated shoe plate includes multiple regions of variable material properties that may conform to an individual runner's gait.

### CROSS REFERENCE TO RELATED APPLICATIONS

This application claims the benefit of U.S. Provisional Application 63/077,610 filed on Sep. 12, 2020, the contents of which are incorporated in its entirety for all purposes.

### FIELD OF THE INVENTION

The present invention relates generally to impact control in foot coverings, and more specifically, but not exclusively, to a variable materials properties structure incorporated into a foot covering and disposed between a foot and an impact surface over which the foot/feet move.

### BACKGROUND OF THE INVENTION

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also be inventions.

Running is a popular form of exercise and offers significant cardiopulmonary, musculoskeletal, metabolic, and vascular benefits. However, every year up to 50% to 60% of runners worldwide sustain repetitive stress and traumatic injuries. Nearly 80% of running injuries are overuse injuries which may be caused or exacerbated by overloading of the musculoskeletal system of lower extremities.

A vertical ground reaction force (VGRF) includes an action of an equal and opposite force between the foot and the ground during the process of running. As the speed of running increases, there is an increase in VGRF. Some estimates consider that recreational and competitive runners have a 70% chance of injury from overuse within a one-year period of time. Injuries of the lower extremities are especially common in runners. When the velocity of running increases, an increase in the amount of VGRF at impact is observed, leading to greater soft tissue stress and bone injuries. Additionally, kinematic asymmetry, often described as the difference between limbs in regard to either kinetic or kinematic parameters, may be an under-appreciated source of injuries, especially for recreational runner. It has been found that when there is asymmetry of 15% or more, which typically increases with faster speeds, there is an increased risk of injury on the lower extremity.

Much of the study of running injury prevention has focused on how to manage (reduce) impact loading. Shock absorption has been defined by the American Society of Testing and Materials (ASTM) as “a reduction of peak force by increasing the time over which the force is applied”. Any energy absorbed by the shoe at impact is not absorbed by the body. Furthermore, a quantity of recoverable energy can be related to the feel of the shoe when the sole is decompressing. As an example, when a “rear foot” runner's foot contacts the ground, the shoe begins to compress. At full compression of the midsole at the rear of the foot, the shoe absorbs its maximum impact energy. Any energy the shoe absorbs is not absorbed by the runner's body. This is considered the maximum energy absorbed by the shoe. As the runner continues through the stride, the VGRF center of pressure (point of force application) propagates anteriorly and the rear of the shoe decompresses. Some of that energy is recovered from the shoe's pushing upward on the plantar surface of the heel. The rest of the energy is lost (may not be recoverable as work) due to a hysteresis of the shoe sole material. The lost energy due to hysteresis and returned energy may be understood as distinct areas under a force-displacement curve.

Maximum energy is the sum of lost energy plus returned energy. The force-displacement curve basically measures the energy into the shoe and the energy returned with for each foot strike as a function of loading and unloading cycle.

In order to reduce a risk of running related injuries, shoe manufacturers have added cushioning to shoe soles in order to reduce impact load. However, many studies show no evidence of reduced running injury rates with increasing amounts of cushioning. As well, there is no current consensus among scholars with regards to the effect of cushioned shoes on the impact force and external loading.

The explanation for this counterintuitive finding may lie in the well-recognized, but poorly understood phenomenon that highly cushioned shoes have a limited ability to reduce impact loading. In fact, some studies have noted even an increase in impact loading when running in shoes with a compliant versus hard midsole. These findings may counter the impact attenuation theory and the result of in vitro mechanical impact studies, both of which indicate a reduction in impact loading with increased cushioning.

Additionally, several studies have explained how maximalist highly padded shoes may alter the body's natural spring-like running mechanics and amplify rather than attenuate impact loading.

An elastic leg behavior is critical to human locomotion. During running, the leg undergoes compression in a first half of the stance while gradually decelerating the body and then recoils in a second half of the stance phase to reaccelerate the body. This cyclic behavior permits efficient force production through a stretch-shortening muscle action and is essential for avoiding mechanically costly high-energy impacts during foot ground contact.

In situ testing has suggested that during running, the elastic tissues within the arch of the human foot (long and short plantar ligaments, the spring ligament, and the plantar aponeurosis) can store 17 joules of elastic energy and contribute significantly to metabolic energy savings. The Achilles tendon is considered to be a primary site of elastic energy storage and release during gait, with contribution of approximately 30-40 joules per step. During running elastic energy is stored within the arch of the foot and the muscles of the lower leg in early stance and released during push off (toe off).

This elastic behavior during running is considered herein as a simple spring-mass system where a leg-spring supports the point mass representing the runner's center of mass.

Importantly, spring-mass studies have shown that running humans maintain the same bouncing movement of the body's center of mass across surfaces with different stiffness by adjusting their leg stiffness during the stance phase. When transitioning from a hard surface to a more compliant surface, a runner's leg becomes stiffer and compresses less to maintain the preferred spring-mass mechanics. Several studies in human running have established that, similar to shoes with different amounts of cushioning, surfaces with different stiffness properties have limited effect on the ground reaction force impact peak (IP) and loading rate (LR).

The underlying mechanism of this phenomenon is not fully understood. Therefore, the runner's legs stiffen and compress less when running in shoes with additional cushion, which in turn might be responsible for countering the impact attenuation effect of the extra cushioning. In fact, as noted earlier, several studies have demonstrated that running in highly cushioned shoes amplifies rather than attenuate impact loading as both impact peak IP and loading rate LR increase.

Overall, studies are inconclusive and do not provide any evidence to suggest that increased cushioning and shock absorption prevents injuries, and as noted, there are concerns that highly cushioned shoes alter gait and running biomechanics negatively.

Therefore, there may be advantages to alternative structures and methods, as compared to conventional shoe cushioning, that may be employed to decrease running injuries.

### BRIEF SUMMARY OF THE INVENTION

Disclosed is a system and method for structures and methods, as compared to conventional shoe cushioning, that may be employed to decrease running injuries. The following summary of the invention is provided to facilitate an understanding of some of the technical features related to variable material properties of foot coverings, and is not intended to be a full description of the present invention. A full appreciation of the various aspects of the invention can be gained by taking the entire specification, claims, drawings, and abstract as a whole. The present invention is applicable to other foot coverings in addition to running shoes.

A method of creating running shoes that DO NOT minimize the body's own natural spring-like mechanism, which diminishes the necessary strain required for proper muscle activation that is crucial to maintenance of healthy muscles, tendons and ligaments.

A method of creating running shoes that DO NOT hinder the body's running performance by impairing mechanosensory feedback and therefore the inherent capacity of the central nervous system to contend with large impact forces via adjustment in leg and foot spring stiffness.

A method of creating running shoes that interfere minimally or not at all with the body's own highly efficient mechanism of recycling elastic and kinetic energy during each foot contact, while also allowing a stable center of mass trajectory.

A method of creating running shoes that do not limit the INHERENT capacity of the human foot to store and return energy via elastic mechanism owing to a reduction in the magnitude of arch and leg compression and recoil.

A method of creating running shoes that DO NOT induce a counter move in the body's natural shock absorbing system by increasing knee, ankle, and foot stiffness, which results in decrease range of motion of the joints and decreased stretch and tension applied to the tendons and ligaments of the leg, foot and ankle, ultimately leading to atrophy, deconditioning, scaring and weakness, making the runner prone to injury and pain.

A method of creating custom running shoes with the ability to normalize (correct) the specific asymmetrical needs of the runner to correct deficient pathological and mechanical gait patterns.

A method of cushioning the shoes without attenuating (or minimally affecting) the runners own muscle activity.

A method of creating running shoes that are highly compliant and flexible at initial impact, progressively becoming more and more non-compliant during the (%) stance phase of gait, and finally exhibiting a high level of non-compliance and stiffness at push off. A shoe that limits impact loads while still allowing more extensive utilization of our own tissues natural spring-like mechanisms.

A shoe that has the ability of maximizing shock absorption to prevent injury, while at the same time maintaining running economy, without compromising one for the other.

A method of creating running shoes that DO NOT eliminate and compromise lower extremity exposure to the natural mechanical stresses and excursion, necessary to maintain a healthy muscle, tendon and ligament structures, mitigating against deconditioning and contracture.

A method of creating running shoes that act like sand on initial contact, like gravel at loading phase, like asphalt at midstance and like concrete at push off. Where the shoe is highly compliant and energy inefficient at initial contact but stiff and energy efficient at push off, with steady and graded variation of compliance and stiffness in the midsole based on trajectory of point of force application during the % stance of gait.

A method of creating running shoes that have “shifting” mechanical properties during (in relation to) the stance phase of gait.

A method of creating running shoes that have a graded non-homogeneous midsole that can be constructed at the nanoscale, microscale, mesoscale and macroscale levels.

A method of creating running shoes that allow locomotion without inducing disuse atrophy.

A method of creating running shoes that can normalize asymmetrical gait patterns related to VGRF and point of force application trajectory.

A method of creating a midsole that provides a “graded physical properties” throughout discretized, segmented, tessellated portions of the midsole based on time (or % of stance phase) and VGRF.

A method of collecting data regarding abnormal gait patters in relation to ‘force footprint’ which is a function of point of force application trajectory and VGRF.

A method of correlating force footprint data with abnormal gait kinematics, providing new diagnostic methods (VMPS diagnostics).

A method of providing targeted therapy based on VMPS diagnostics.

A method of using biofeedback through wearable technologies to correct abnormal gait patterns and reduce running injuries.

A method of creating polymers for running shoes with graded non-homogenous (physical) mechanical properties at the nanoscale, microscale, mesoscale and macroscale levels.

A method of creating foam for running shoes with graded non-homogenous (physical) mechanical properties at the nanoscale, microscale, mesoscale and macroscale levels.

A method of providing enhanced POC point of care diagnostics by embedding tissue microchips to further evaluate muscle/tendon tissues implicated by VMPS diagnostics.

A foot covering for a human foot, including a sole; and an upper configured to couple the sole to the human foot; and a variable materials properties structure incorporated into the sole.

The foot covering wherein a person wearing the foot covering on the human foot during a movement over a surface includes a gait pattern and wherein the variable materials properties structure includes a gait adapting arrangement responsive to the gait pattern.

The foot covering wherein the gait pattern includes a plurality of gait phases and wherein the gait adapting arrangement includes a first set of materials properties for the variable materials properties structure during a first gait phase and further includes a second set of materials properties for the variable materials properties structure during a second gait phase, the first gait phase different from the second gait phase and the first set of materials properties different from the second set of materials properties.

The foot covering wherein the sole includes an outsole and a inner sole disposed between the outsole and the human foot, the variable materials properties structure disposed in the inner sole.

The foot covering wherein the variable materials properties structure includes a distributed nonhomogeneous properties gradient over an expanse of the variable materials properties structure.

The foot covering wherein the distributed nonhomogeneous properties gradient is configured to correspond to an individual's specific footprint which is a function of a point of force application trajectory and a vertical ground reaction force.

The foot covering wherein the variable materials properties structure includes a set of gait-characterizing sensors responsive to the gait pattern during the movement over the surface.

The foot covering wherein the gait pattern results from a biomechanical condition for the human, the biomechanical condition including a degradation of the gait pattern with respect to a standard gait and wherein the set of gait-characterizing sensors are configured for an evaluation of the biomechanical condition during the movement.

The foot covering wherein the evaluation is configured to define a second variable materials properties structure configured to reduce the degradation.

A pair of foot coverings for a pair of human feet, including a pair of soles, one sole for each foot of the pair of feet; and a pair uppers, each configured to uniquely couple one of the soles to one of the foots; and a first variable materials properties structure incorporated into a first one of the soles; and a second variable materials properties structure incorporated into a second one of the soles, the variable materials properties structures providing different variable materials properties.

A method for ameliorating a set of negative gait components of a gait pattern of a person moving over a surface while wearing a set of foot coverings, including incorporating at least one variable materials properties structure into a sole of at least one of the foot coverings of the set of foot coverings; and compensating one or more of the gait components of the set negative gait components using the variable materials properties structure.

The method wherein the compensating step includes measuring elements of the set of negative gait components during movement contributing to a degraded gait, and customizing the variable materials properties structure to decrease biomechanical contributions to the degraded gait.

A method for evaluating a gait pattern of a person moving over a surface while wearing a set of foot coverings, including incorporating at least one variable materials properties structure into a sole of at least one of the foot coverings of the set of foot coverings; and characterizing a set of gait elements of the gait pattern using the variable materials properties structure.

Any of the embodiments described herein may be used alone or together with one another in any combination. Inventions encompassed within this specification may also include embodiments that are only partially mentioned or alluded to or are not mentioned or alluded to at all in this brief summary or in the abstract. Although various embodiments of the invention may have been motivated by various deficiencies with the prior art, which may be discussed or alluded to in one or more places in the specification, the embodiments of the invention do not necessarily address any of these deficiencies. In other words, different embodiments of the invention may address different deficiencies that may be discussed in the specification. Some embodiments may only partially address some deficiencies or just one deficiency that may be discussed in the specification, and some embodiments may not address any of these deficiencies.

Other features, benefits, and advantages of the present invention will be apparent upon a review of the present disclosure, including the specification, drawings, and claims.

### BRIEF DESCRIPTION OF THE DRAWINGS

The accompanying figures, in which like reference numerals refer to identical or functionally-similar elements throughout the separate views and which are incorporated in and form a part of the specification, further illustrate the present invention and, together with the detailed description of the invention, serve to explain the principles of the present invention.

FIG. 1 illustrates a vertical ground reaction force versus time during stance phase of running gait cycle including an impact peak, a loading rate, and an active peak;

FIG. 2 illustrates samples of force-displacement curves of cushioned running shoes including loading and unloading with each foot strike;

FIG. 3 illustrates samples of force-displacement curves of cushioned running shoes including maximum energy absorbed=lost energy+returned energy;

FIG. 4 illustrates an elastic behavior of running as a simple spring-mass system, where a leg-spring system supports a runner's center of mass (CoM);

FIG. 5 illustrates heel strike (rearfoot) versus midfoot or forefoot strikes (non-rearfoot);

FIG. 6 illustrates a foot-shoe system having two springs in-series;

FIG. 7 illustrates a shock absorption and resilience of current running shoes with a mid-level stiffness and compliance;

FIG. 8 illustrates a very stiff, highly non-compliant, and energy efficient midsole;

FIG. 9 illustrates a very soft, highly compliant and energy inefficient midsole;

FIG. 10 illustrates a uniform and unchanging shock absorption and resilience throughout a tessellated/segmented foot plate, for example every single quadrant of a segmented/tessellated current midsole has the exact same level of stiffness, compliance, shock absorption and resilience;

FIG. 11 illustrates changing force-displacement curves over (% of stance phase) of VMPS;

FIG. 12 illustrates graded variation in (shock absorption) during a stance phase of running gait cycle;

FIG. 13 illustrates cushioned running shoe midsoles including compliance, stiffness, shock absorption, resilience that are all constant, and unchanging over time during the stance phase of the running gait cycle (% stance phase);

FIG. 14 illustrates a variable material properties shoe (VMPS) midsole including graded compliance, stiffness, shock absorption, resilience over (%) stance phase having varied, graded, non-homogenous structure of the “midsole” producing certain function during stance phase of gait;

FIG. 15 illustrates a graded VMPS midsole having different quality and quantity of shock absorption as compared with highly cushioned shoe midsoles;

FIG. 16 illustrates a graded VMPS midsole having different quality and quantity of resilience as compared with highly cushioned shoe midsoles;

FIG. 17 illustrates an arrangement of pressure sensors in mini regions of a segmented (tessellated) foot plate and a presentation of anterior, posterior, medial and lateral aspects of a foot plate;

FIG. 18 illustrates a point of force application trajectory including a posterior anterior direction for a heel strike (rear foot) runner and lateral-medial direction for a midfoot strike (non-rear foot) runner;

FIG. 19 illustrates a force versus displacement curves of VMPS midsoles showing varying graded stiffness from low to high based on % of stance phase, responsive to point of force trajectory including circles represent stiffness (larger circles representing increasing levels of stiffness; increasing levels of stiffness and associated (force-displacement curves) complementary to an anterolateral to anteromedial point of force application trajectory; and increasing levels of stiffness and associated (force-displacement curve) complementary to a posterior to anterior point of force application trajectory;

FIG. 20 illustrates a monotonic graded non-homogenous increase in stiffness in VMPS midsole from posterior to anterior with larger circles representing increasing levels of stiffness;

FIG. 21 illustrates a monotonic graded non-homogenous increase in stiffness in VMPS midsole from anterolateral to anteromedial with larger circles representing increasing levels of stiffness;

FIG. 22 illustrates an asymmetrical gait with respect to point of force application trajectory with a left VMPS midsole becoming progressively stiffer from posterior to anterior, with a right VMPS midsole becoming progressively stiffer from anterolateral to anteromedial;

FIG. 23 illustrates an asymmetrical gait with respect to VGRF with a stiffness profile of a left VMPS midsole progressing from moderate to high, with a stiffness profile of a right VMPS midsole progressing from mild to moderate;

FIG. 24 illustrates an atypical hybrid point of force application trajectory from posterolateral to anteromedial;

FIG. 25-FIG. 28 illustrate steps in creation of a left VMPS midsole for a runner with a high VGRF left heel strike pattern progressing from posterolateral to anteromedial;

FIG. 25 illustrates segmentation of foot plate with mini quadrant force sensors;

FIG. 26 illustrates a measurement of a “force footprint”=VGRF and point of force application trajectory;

FIG. 27 illustrates a superimposition of a point of force application on a segmented foot plate; and

FIG. 28 illustrates creation of a graded stiffness profile complementary to point of force trajectory;

FIG. 29-FIG. 32 illustrate steps in creation of a moderate VGRF right midfoot strike pattern progressing from anterolateral to anteromedial;

FIG. 29 illustrates segmentation of foot plate with mini quadrant force sensors;

FIG. 30 illustrates a measurement of a “force footprint”=VGRF and point of force application trajectory;

FIG. 31 illustrates a superimposition of a point of force application on a segmented foot plate; and

FIG. 32 illustrates creation of a graded stiffness profile complementary to point of force trajectory;

FIG. 33 illustrates a specialized microchips embedded in major lower extremity muscle groups, including quadriceps, hamstrings, gastrocsoleious, and plantar fascia;

FIG. 34 illustrates a biofeedback modality including an instantaneous feedback regarding runner's VGRF and point of force application trajectory sensed by mini-quadrant force sensors in VMPS midsole and transmitted to runner's smart glasses wirelessly, and displayed graphically;

FIG. 35 illustrates a force versus displacement curve for typical midsole foams used in running shoes, showing loading and unloading of the foam, with energy lost as hysteresis, energy returned as resilience, maximum energy absorbed by the shoe as a sum of lost energy and returned energy; and

FIG. 36 illustrates polymer modification including branching, crosslinking, and crystals.

### DETAILED DESCRIPTION OF THE INVENTION

Embodiments of the present invention provide a system and method for structures and methods, as compared to conventional shoe cushioning, that may be employed to decrease running injuries. The following description is presented to enable one of ordinary skill in the art to make and use the invention and is provided in the context of a patent application and its requirements.

Various modifications to the preferred embodiment and the generic principles and features described herein will be readily apparent to those skilled in the art. Thus, the present invention is not intended to be limited to the embodiment shown but is to be accorded the widest scope consistent with the principles and features described herein.

### Definitions

Unless otherwise defined, all terms (including technical and scientific terms) used herein have the same meaning as commonly understood by one of ordinary skill in the art to which this general inventive concept belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

The following definitions apply to some of the aspects described with respect to some embodiments of the invention. These definitions may likewise be expanded upon herein.

As used herein, the term “or” includes “and/or” and the term “and/or” includes any and all combinations of one or more of the associated listed items. Expressions such as “at least one of,” when preceding a list of elements, modify the entire list of elements and do not modify the individual elements of the list.

As used herein, the singular terms “a,” “an,” and “the” include plural referents unless the context clearly dictates otherwise. Thus, for example, reference to an object can include multiple objects unless the context clearly dictates otherwise.

Also, as used in the description herein and throughout the claims that follow, the meaning of “in” includes “in” and “on” unless the context clearly dictates otherwise. It will be understood that when an element is referred to as being “on” another element, it can be directly on the other element or intervening elements may be present therebetween. In contrast, when an element is referred to as being “directly on” another element, there are no intervening elements present.

As used herein, the term “set” refers to a collection of one or more objects. Thus, for example, a set of objects can include a single object or multiple objects. Objects of a set also can be referred to as members of the set. Objects of a set can be the same or different. In some instances, objects of a set can share one or more common properties.

As used herein, the term “adjacent” refers to being near or adjoining. Adjacent objects can be spaced apart from one another or can be in actual or direct contact with one another. In some instances, adjacent objects can be coupled to one another or can be formed integrally with one another.

As used herein, the terms “connect,” “connected,” and “connecting” refer to a direct attachment or link. Connected objects have no or no substantial intermediary object or set of objects, as the context indicates.

As used herein, the terms “couple,” “coupled,” and “coupling” refer to an operational connection or linking. Coupled objects can be directly connected to one another or can be indirectly connected to one another, such as via an intermediary set of objects.

The use of the term “about” applies to all numeric values, whether or not explicitly indicated. This term generally refers to a range of numbers that one of ordinary skill in the art would consider as a reasonable amount of deviation to the recited numeric values (i.e., having the equivalent function or result). For example, this term can be construed as including a deviation of ±10 percent of the given numeric value provided such a deviation does not alter the end function or result of the value. Therefore, a value of about 1% can be construed to be a range from 0.9% to 1.1%.

As used herein, the terms “substantially” and “substantial” refer to a considerable degree or extent. When used in conjunction with an event or circumstance, the terms can refer to instances in which the event or circumstance occurs precisely as well as instances in which the event or circumstance occurs to a close approximation, such as accounting for typical tolerance levels or variability of the embodiments described herein.

As used herein, the terms “optional” and “optionally” mean that the subsequently described event or circumstance may or may not occur and that the description includes instances where the event or circumstance occurs and instances in which it does not.

As used herein, the term “size” refers to a characteristic dimension of an object. Thus, for example, a size of an object that is spherical can refer to a diameter of the object. In the case of an object that is non-spherical, a size of the non-spherical object can refer to a diameter of a corresponding spherical object, where the corresponding spherical object exhibits or has a particular set of derivable or measurable properties that are substantially the same as those of the non-spherical object. Thus, for example, a size of a non-spherical object can refer to a diameter of a corresponding spherical object that exhibits light scattering or other properties that are substantially the same as those of the non-spherical object. Alternatively, or in conjunction, a size of a non-spherical object can refer to an average of various orthogonal dimensions of the object. Thus, for example, a size of an object that is a spheroidal can refer to an average of a major axis and a minor axis of the object. When referring to a set of objects as having a particular size, it is contemplated that the objects can have a distribution of sizes around the particular size. Thus, as used herein, a size of a set of objects can refer to a typical size of a distribution of sizes, such as an average size, a median size, or a peak size.

Running is a popular form of exercise and offers significant cardiopulmonary, musculoskeletal, metabolic and vascular benefits. However, every year up to 50% to 60% of runners worldwide sustain repetitive stress and traumatic injuries. Nearly 80% of running injuries are overuse injuries which are caused by overloading of the musculoskeletal system of lower extremities.

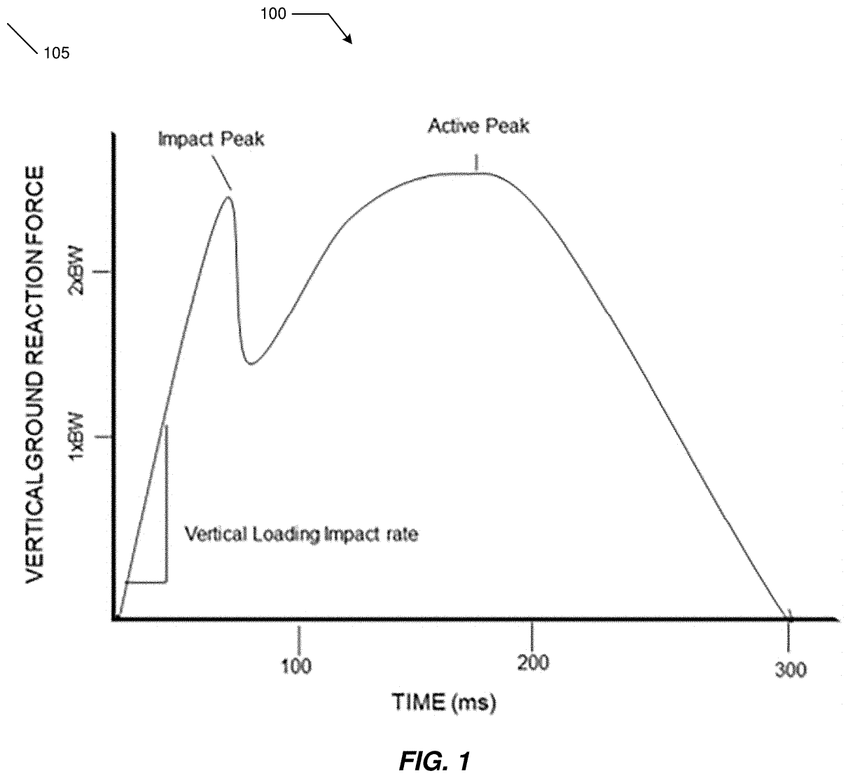

The vertical ground reaction force VGRF is the action of an equal and opposite force between the foot and the ground during the process of running. As the speed of running increases, there is an increase in VGRF. It has been estimated that recreational and competitive runners have a 70% chance of injury from overuse within a one-year period of time. Injuries of the lower extremities are especially common in runners. When the velocity of running increases, an increase in the amount of VGRF at impact is observed, leading to greater soft tissue stress and bone injuries. Additionally, kinematic asymmetry, often described as the difference between limbs in regard to either kinetic or kinematic parameters, appears to be an under-appreciated source of injuries, especially in the recreational runner. It has been found that when there is asymmetry of 15% or more, which typically increases with faster speeds, there is an increased risk of injury on the lower extremity. FIG. 1 illustrates a vertical ground reaction force versus time during stance phase of running gait cycle including an impact peak, a loading rate, and an active peak.

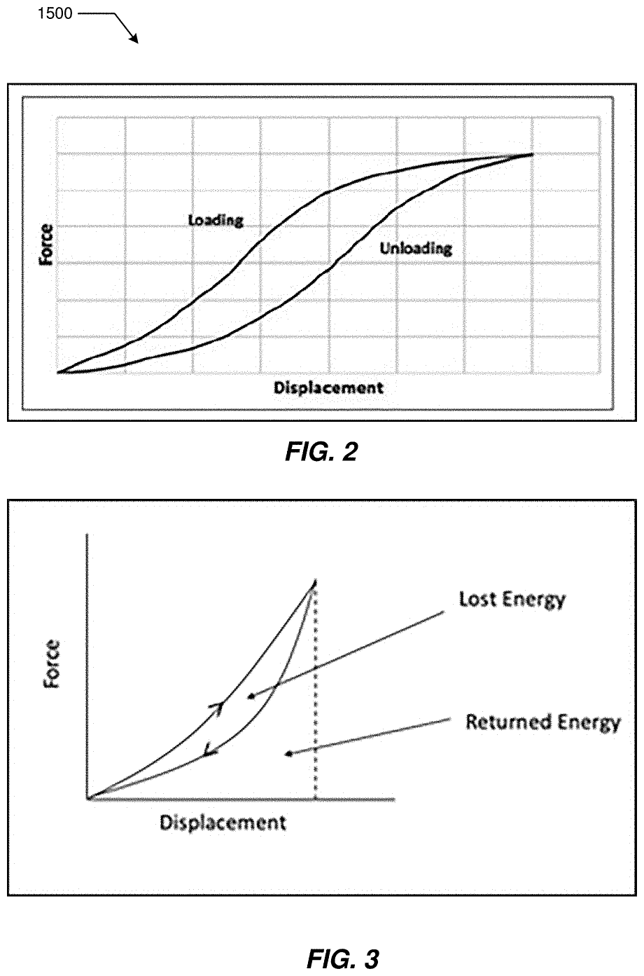

Much of the study of running injury prevention has focused on how to manage (reduce) impact loading. Shock absorption has been defined by the American Society of Testing and Materials (ASTM) as “the reduction of peak force by increasing the time over which the force is applied”. Any energy absorbed by the shoe at impact is not absorbed by the body. Furthermore, the quantity of recoverable energy can be related to the feel of the shoe when the sole is decompressing. As an example, when a Rear foot runner's foot contacts the ground, the shoe begins to compress. At full compression of the midsole at the rear of the foot, the shoe absorbs its maximum impact energy. Any energy the shoe absorbs is not absorbed by the runner's body. This is considered the maximum energy absorbed by the shoe. As the runner continues through the stride, the VGRF center of pressure (point of force application) propagates anteriorly and the rear of the shoe decompresses. Some of that energy is recovered from the shoe's pushing upward on the plantar surface of the heel. The rest of the energy is lost (i.e. not recoverable as work) due to hysteresis of the shoe sole material. The lost energy due to hysteresis and returned energy are shown as distinct areas under the force-displacement curves in FIG. 2-FIG. 3.

Maximum energy is the sum of lost energy plus returned energy. The force-displacement curve basically measures the energy into the shoe and the energy returned with for each foot strike as a function of loading and unloading cycle. FIG. 2 illustrates samples of force-displacement curves of cushioned running shoes including loading and unloading with each foot strike. FIG. 3 illustrates samples of force-displacement curves of cushioned running shoes including maximum energy absorbed=lost energy+returned energy.

In order to reduce the risk of running related injuries, shoe manufacturers have added cushioning to shoe soles in order to reduce impact load. However, many studies show no evidence of reduced running injury rates with increasing amounts of cushioning. As well, there is no current consensus among scholars with regards to the effect of cushioned shoes on the impact force and external loading.

The explanation for this counterintuitive finding may lie in the well-recognized, but poorly understood phenomenon that highly cushioned shoes have a limited ability to reduce impact loading. In fact, some studies have noted even an increase in impact loading when running in shoes with a compliant versus hard midsole. These findings counter the impact attenuation theory and the result of in vitro mechanical impact studies, both of which indicate a reduction in impact loading with increased cushioning.

Additionally, several studies have explained how maximalist highly padded shoes alter the body's natural spring-like running mechanics and amplify rather than attenuate impact loading.

The elastic leg behavior is critical to human locomotion. During running, the leg undergoes compression in the first half of the stance while gradually decelerating the body and then recoils in the second half of the stance phase to reaccelerate the body. This cyclic behavior permits efficient force production through a stretch-shortening muscle action and is essential for avoiding mechanically costly high-energy impacts during foot ground contact.

In situ testing has suggested that during running, the elastic tissues within the arch of the human foot (long and short plantar ligaments, the spring ligament, and the plantar aponeurosis) can store 17 joules of elastic energy and contribute significantly to metabolic energy savings. The Achilles tendon is considered to be the primary site of elastic energy storage and release during gait, with contribution of approximately 30-40 joules per step. During running elastic energy is stored within the arch of the foot and the muscles of the lower leg in early stance and released during push off (toe off).

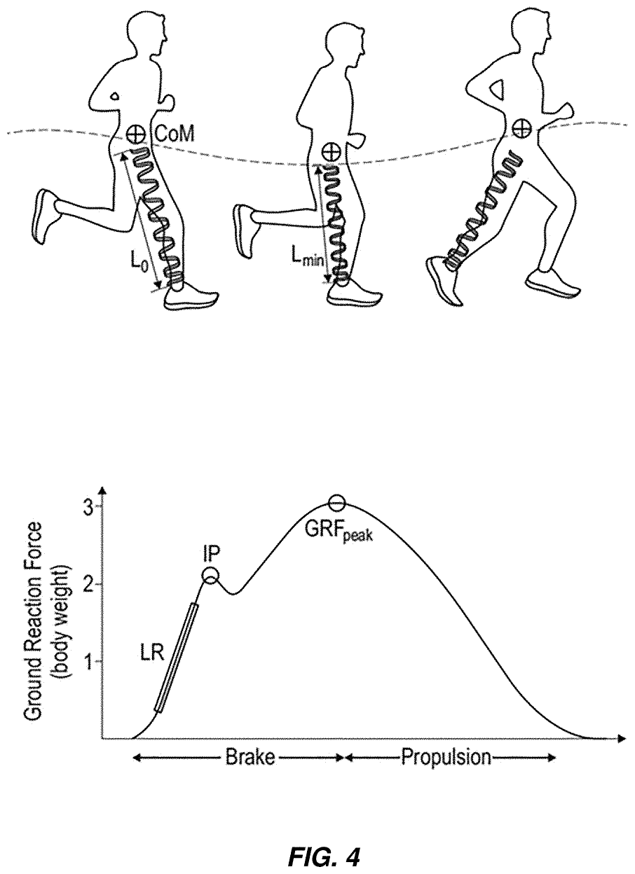

The elastic behavior during running can be described as a simple spring-mass system where a leg-spring supports the point mass representing the runner's center of mass. FIG. 4 illustrates an elastic behavior of running as a simple spring-mass system, where a leg-spring system supports a runner's center of mass (CoM).

Importantly, spring-mass studies have shown that running humans maintain the same bouncing movement of the body's center of mass across surfaces with different stiffness by adjusting their leg stiffness during the stance phase. When transitioning from a hard surface to a more compliant surface, a runner's leg becomes stiffer and compresses less to maintain the preferred spring-mass mechanics. Several studies in human running have established that, similar to shoes with different amounts of cushioning, surfaces with different stiffness properties have limited effect on the ground reaction force impact peak (IP) and loading rate (LR).

The underlying mechanism of this phenomenon is not fully understood. Therefore, the runner's legs stiffen and compress less when running in shoes with additional cushion, which in turn might be responsible for countering the impact attenuation effect of the extra cushioning. In fact, as noted earlier, several studies have demonstrated that running in highly cushioned shoes amplifies rather than attenuate impact loading as both impact peak IP and loading rate LR increase.

Overall, studies are inconclusive and do not provide any evidence to suggest that increased cushioning and shock absorption prevents injuries, and as noted, there are concerns that highly cushioned shoes alter gait and running biomechanics negatively.

Therefore, there is a need for alternative methods, other than standard cushioned shoes, that can be employed to decrease running injuries.

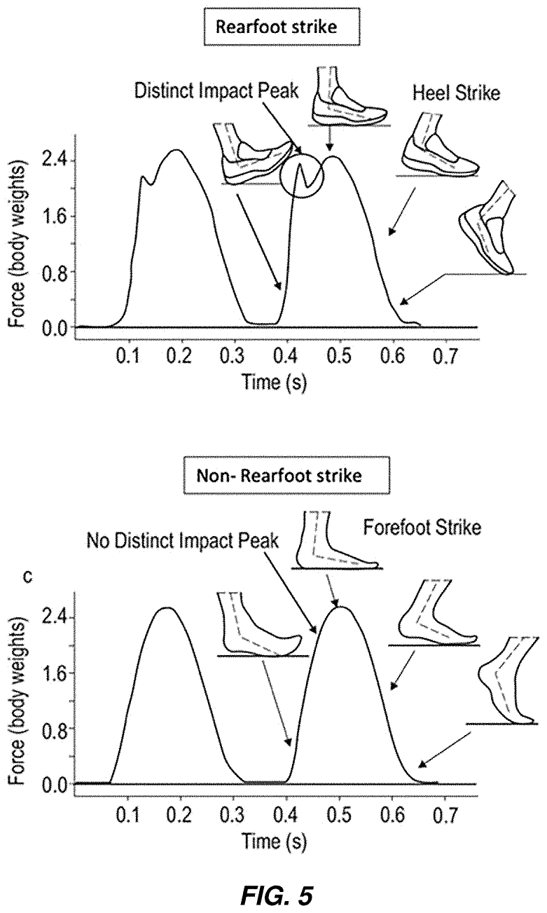

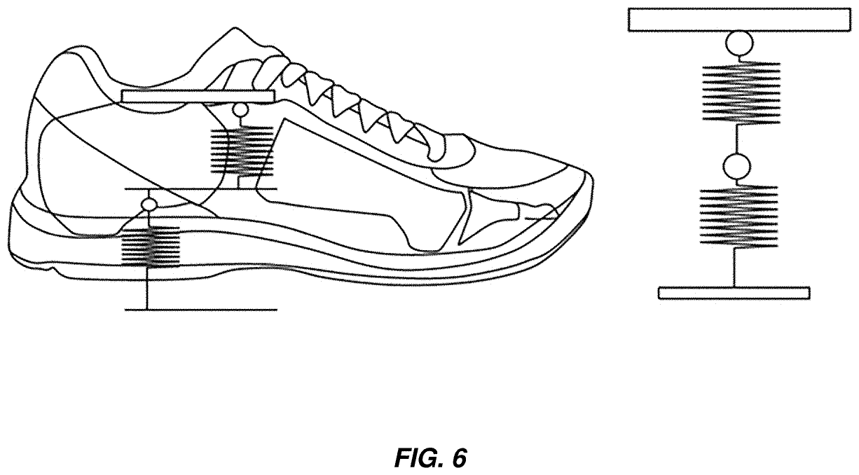

Runners typically have different habits when it comes to foot strike patterns. Runners strike the ground with their forefoot, midfoot or hind foot. These strike patterns are commonly referred to as Rearfoot (RF) or Non-Rear foot (NRF) strike. NRF includes both midfoot and forefoot strike patterns. FIG. 5 illustrates heel strike (rearfoot) versus midfoot or forefoot strikes (non-rearfoot).

It is notable that the strike pattern of an individual runner (foot kinematics) can differ with different running speeds. Additionally, the strike patterns may alter within the same individual runner's left and right feet, potentially based on chronic musculoskeletal injuries and mechanical deficiencies.

There is a common belief that highly cushioned shoes over the last several decades have increased the incidence of heel strike (RF) running.

Conversely, several authors have hypothesized that decreasing the cushioning of the shoe would promote a more midfoot (NRF) strike pattern, similar to what is seen in barefoot running, where the foot and leg's intrinsic energy absorbing mechanisms work naturally to absorb and release energy.

Barefoot runners typically avoid heel strike (RF) running. Barefoot runners are typically midfoot (NRF) runners and use the body's natural spring like mechanisms more effectively, including the windlass mechanism of the foot, Achilles tendon and the lower extremity muscles to store and release energy during a run.

So far, there is no clear consensus that running kinematics and habituation has ever been shown to change (i.e. converting from RF to NRF or vice versa) based on shoe cushioning.

The capacity to store and return energy in the muscles, tendons, ligaments and fascia of the human foot, ankle and leg is crucial to human running economy. Some have suggested that modern running shoes actually impede leg and foot spring-like function by reducing the contributions from the leg and foot musculature (muscle activation and excursion/tensioning of tendons and ligaments).

Even though shoes have provided shock absorption for human feet for running, contemporary running shoes were not invented until 1970s and have evolved in parallel with the surge in popularity of running as a recreational sport.

There is some concern that over the last several decades, shoe manufacturer's focus on the highly cushioned shoe has actually jeopardized the recreational runner, simply because any shoe that minimizes the body's own natural spring-like mechanism diminishes the necessary strain required for proper muscle activation that is crucial to maintenance of healthy muscles, tendons and ligaments.

A defining characteristic of the modern running shoe is the thick viscoelastic midsole that is designed to compress and rebound when cyclically loaded and unloaded during running. This design feature, referred to as cushioning allows the shoe to function in a similar spring like manner to the lower limb and foot, absorbing the potentially harmful impact transients that are encountered when foot impacts the ground, while also returning some of this energy to aid power generation for propulsion. Another key feature is the contoured midsole, designed to provide external support and reduce excessive strain on the muscles and ligaments of the foot.

However, despite the huge financial investment in the development of the running shoes, running injury rate remain relatively unchanged over the last 40 years, leading some to question the efficacy of modern running shoes in preventing injury.

In addition to the fact that an apparent reliance on a shoe to attenuate impact may reduce the runner's own muscle activity predisposing to injury; some authors have suggested that cushioned midsoles may actually hinder our running performance by impairing mechano-sensory feedback and therefore, the inherent capacity of the central nervous system to contend with large impact forces via adjustment in leg and foot spring stiffness.

During the stance phase, the lower limbs of human runners behave in a spring like manner, compressing and recoiling via a sequence of hip, knee, ankle flexion; then extension in phase with the increasing and decreasing magnitude of the vertical ground reaction force. The highly efficient mechanism allows recycling of elastic and kinetic energy during each foot contact while also allowing a relatively stable center of mass trajectory. The central nervous system has the capacity to adjust the stiffness of the lower limb in order to minimize center of mass vertical motion when running across terrains with varying undulations and compliance.

Several studies have confirmed the notion that running shoes actually limit the capacity for the foot to store and return energy via elastic mechanism owing to a reduction in the magnitude of arch and leg compression and recoil. This cushioning and support impair foot-spring function, with a likely consequence of reduced activation from muscles that support the arch, foot and leg, leading to their weakness and disuse atrophy.

Some studies have shown that when a runner wears a shoe with viscoelastic midsole (highly cushioned) the shoe will behave as and additional spring, also with a given stiffness and the two (runner's foot and shoe) form a foot-shoe system that has a stiffness that is dependent on the configuration of the two springs. Therefore, the running shoes act as an additional spring in-series with the foot and leg. FIG. 6 illustrates a foot-shoe system having two springs in-series.

This added in-series spring to the system induces a counter move in the body's natural shock absorbing system by increasing knee, ankle and foot stiffness, which results in decreased range of motion of the joints and decreased stretch and tension applied to the tendons and ligaments of the leg, foot and ankle, ultimately leading to atrophy, deconditioning, scaring and weakness, making the runner prone to injury and pain.

Barefoot runners conversely are more likely to strike the ground with a forefoot first contact, allowing the body to effectively damp the large impact transients via controlled dorsiflexion of the ankle and associated stretch of the arch, Achilles tendon and leg muscles.

Barefoot and midfoot (NRF) runners, in general, utilize the human body's natural shock absorbing mechanisms more effectively than rearfoot (RF) runners, because the muscles, ligaments, tendons and fascia of the lower extremity are more effectively exposed to proper stress and tension, necessary for development and maintenance of strong and healthy tissues, which therefore mitigates against disuse atrophy, contracture and injury.

With construction of a maximalist cushioned shoe, the manufacturers have two distinct goals of increasing running economy and decreasing running injuries. The first goal is to increase running economy such that a competitive elite athlete can run faster with less energy use. For example, there appears to be tremendous excitement about a manufacturer's new “springy” running shoe that allows 4% more energy return (higher resilience) than that of the previous marathon running shoes. This enhancement allows the professional athlete to run a “sub-two hour” marathon or to shave 0.1 seconds of her sprint time. However, the second goal of reducing injuries for the recreational runner is significantly more important. Achievement of this goal can have tremendous sociological, health and economic ramifications, particularly since so many humans are limited in movement (due to pain and injury) which substantially affects their metabolic, cardiovascular and musculoskeletal health.

There is a large body of scientific work funded by multibillion-dollar shoe companies that propound the benefits of the cushioned running shoe for the recreational athlete. However, as we have discussed, the enhanced cushions maybe making the recreational runner, by far the major consumer (99.999%) of the running shoes, weaker and stiffer and more prone to injury.

In addition to highly cushioned viscoelastic shoes, some clinicians prescribe orthotics and ‘stability shoes’ which tend to produce solid blocks of stiffer material under the medial arch of the foot or heel in order to support the midfoot or hindfoot from “collapse” and “too much rotation”, in order to minimize injury and pain. All of these modifications ultimately decrease the natural stresses to the foot and ankle required for healthy muscle development and maintenance. Lastly, some shoe manufactures have used a “rocker bottom” configuration to provide comfort; this concept similar to other methods, diminishes natural motion of the foot and leg, producing weakness and stiffness over time.

An additional problem to note, especially in the recreational runner, and which mostly been ignored by the shoe manufacturers, is the fact that recreational runners frequently exhibit asymmetrical foot strike patterns. For example, a runner may have a RF strike for her left foot and a NRF strike for her right foot, or have a higher VGRF on one side than the other. This phenomena may be related to chronic deconditioning related to previous unilateral injuries.

Recreational runners generally have different goals than the elite athletes. An average runner may want to run two miles per day, three times per week for a total of six miles per week, at an average speed of 10 minutes per mile. Her goal is simply to be healthy, remain active and avoid injury.

Conversely, a professional runner may want to run 70 to 100 miles per week, 10 miles per session, at an average high speed 6 minutes per mile, working out 6 hours per day. Her goal is to shave 2 minutes off her marathon time, or shave 0.1 second off her sprint time.

These two archetypical runners live different lifestyles and have different needs with respect to shoe wear. It is likely that these two types of runners need completely different set of attributes in their running shoes.

As we have discussed, there is minimal if any evidence that traditional running shoe prescription (i.e. rearfoot pronation control, midsole hardness) and/or highly cushioned shoes reduce risk of injury.

We therefore ask two questions: 1. Can we build custom shoes that meets the specific individual needs of these two types of runners? 2. Is there a reliable way to correct deficient mechanical and/or pathological gait kinematics of a runner? 3. Is there a way to have cushioning in the shoes without attenuating the runners own muscle activity?

For example, if a high VGRF foot strike on one side is causing knee and back pain, how do we alter it to a low VGRF foot strike to ameliorate the knee and back pain?

Likewise, how do we construct custom shoes that allow use of the runner's own tissues (muscles, tendons and ligaments) so as to avoid disuse atrophy, deconditioning, contracture and propensity to injury?

Four distinct concepts are proposed to help runners avoid injury and remain mobile. The first and most important principle involves a special midsole cushion that allows better activation and use of the runner's own tissues to avoid deconditioning and injury. The second principle involves awareness of the individual's asymmetric gait kinematics and provision for a “normalizing” custom shoe that is responsive to the runner's individual needs. The third principle provides an entirely new point of care (POC) diagnostics criteria based on force footprint evaluation of gait kinematics; which correlate footprints with abnormal gait as result of old injuries. “Force footprint” is determined by a combined evaluation of the VGRF magnitude and the trajectory of point of force application during (%) stance phase. The fourth principle provides for meaningful and targeted therapies, including biofeedback, based on new POC diagnostic criteria noted.

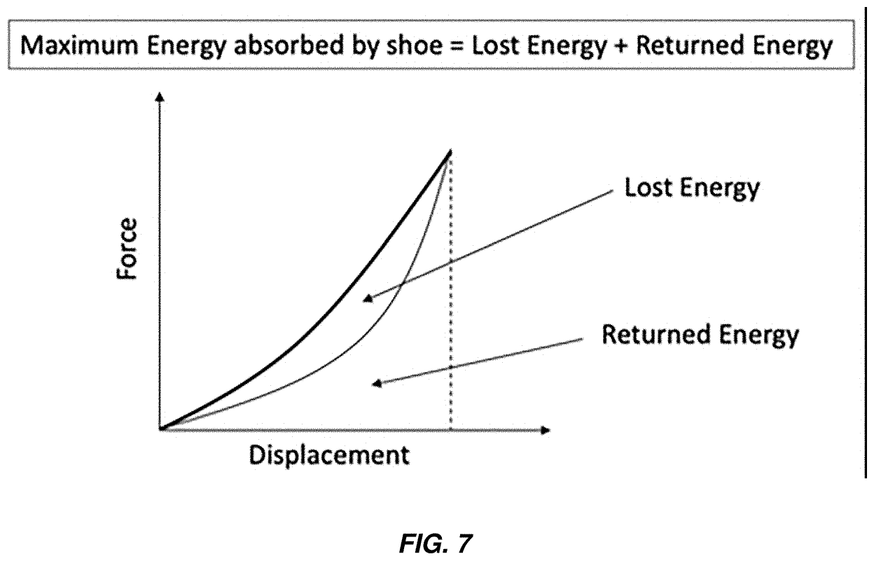

The proprietary polymer foams that are used for highly cushioned shoes are uniform throughout the whole midsole, at nanoscale, mesoscale and microscale levels; and are developed in such a way to provide optimum shock absorption and resilience (energy return), which necessitates a compromise in the level of stiffness of the midsole. A stiff running shoe is highly energy efficient but has poor compliance resulting in low shock absorption. Conversely, a very compliant running shoe provides good shock absorption but is energy inefficient. FIG. 7 illustrates a shock absorption and resilience of current running shoes with a mid-level stiffness and compliance.

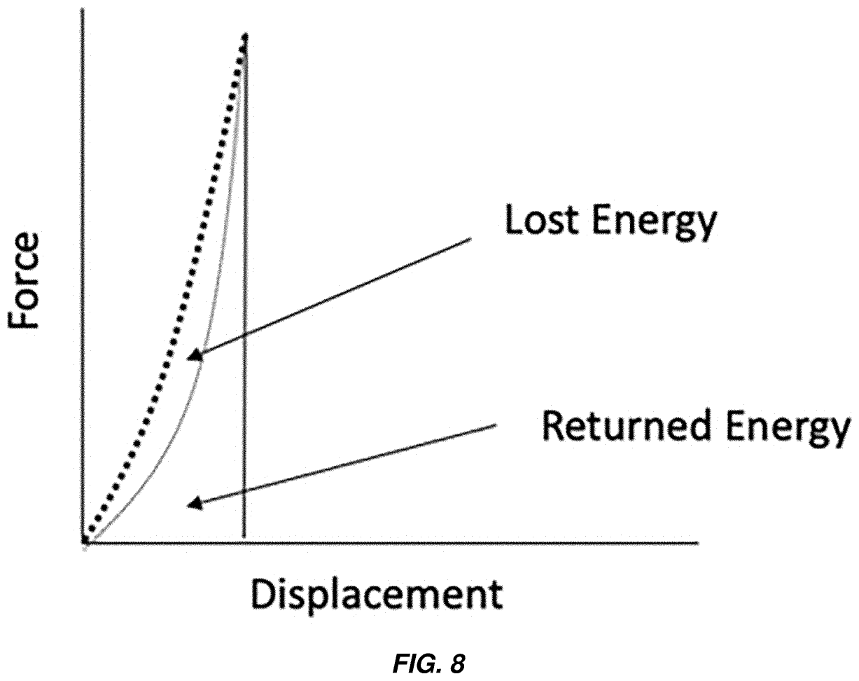

However, these midsoles could be created to be very stiff (high modulus of elasticity), very energy efficient, and very non-compliant. FIG. 8 illustrates a very stiff, highly non-compliant, and energy efficient midsole.

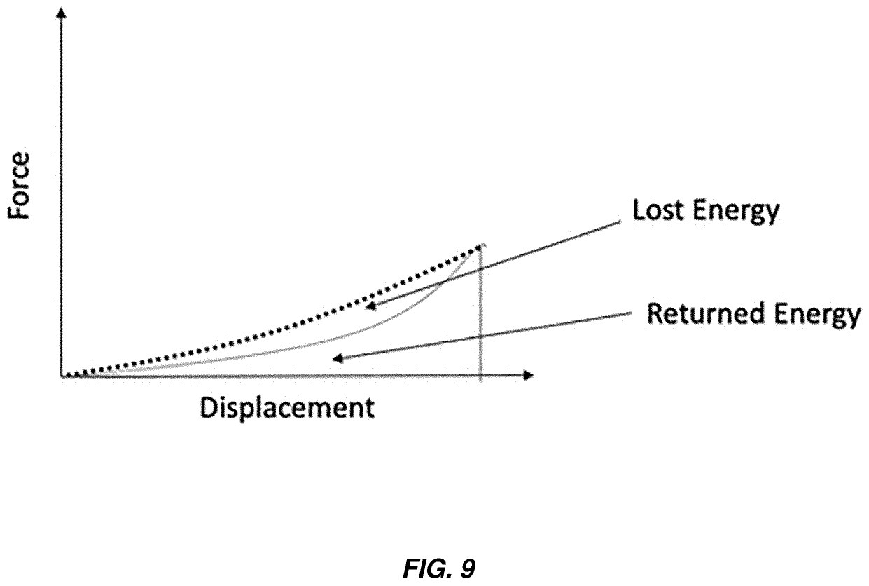

Alternatively, the midsoles could be constructed to be very soft, flexible, compliant and highly energy inefficient. FIG. 9 illustrates a very soft, highly compliant and energy inefficient midsole.

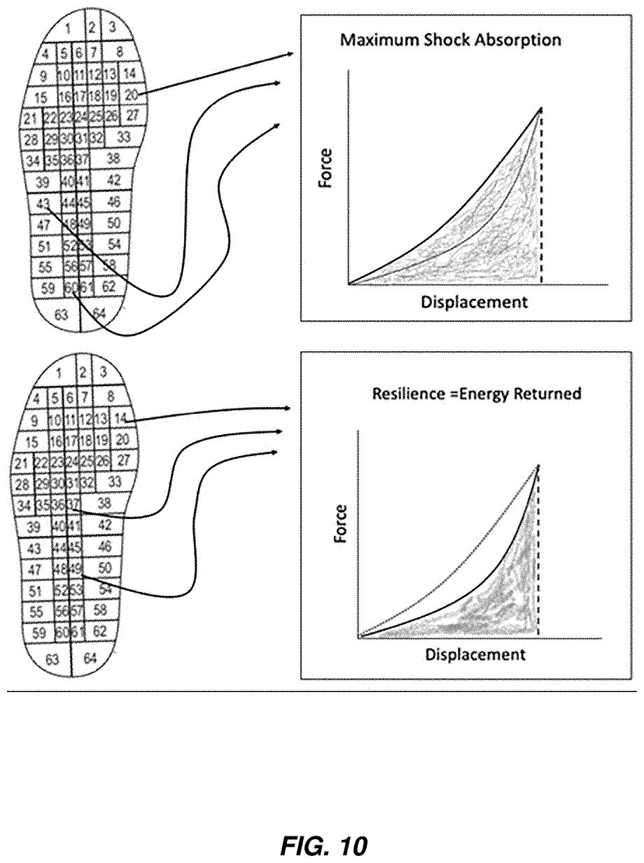

Therefore, the structure of current running shoes is uniform at microscale levels and has a constant stiffness profile throughout the midsole, with a constant shock absorption and resilience values; such that every discretized, segmented, tessellated aspect of the midsole exhibits the exact same force-displacement characteristics, shock absorption and resilience. FIG. 10 illustrates a uniform and unchanging shock absorption and resilience throughout a tessellated/segmented foot plate, for example every single quadrant of a segmented/tessellated current midsole has the exact same level of stiffness, compliance, shock absorption and resilience.

In order to address the questions posed we propose the concept of a variable material properties shoe VMPS.

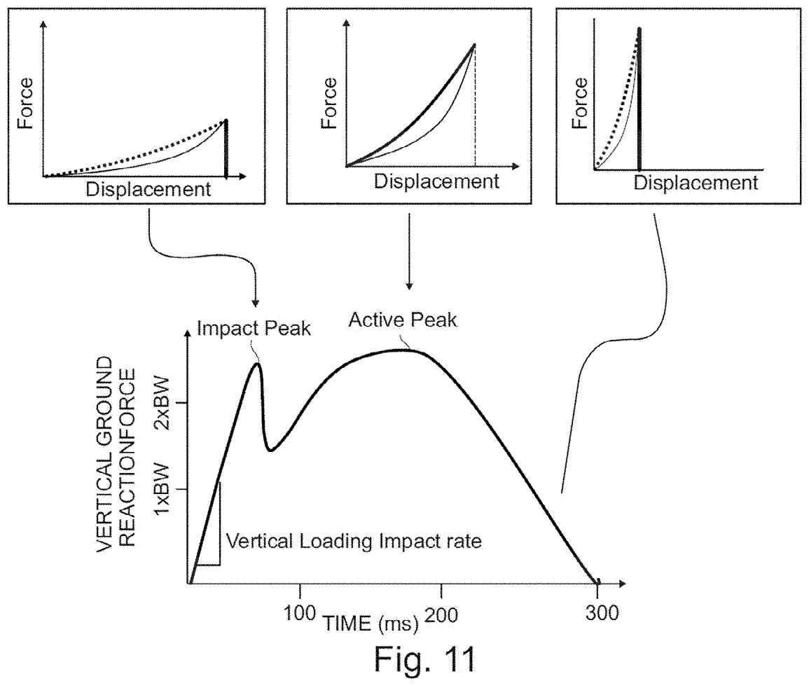

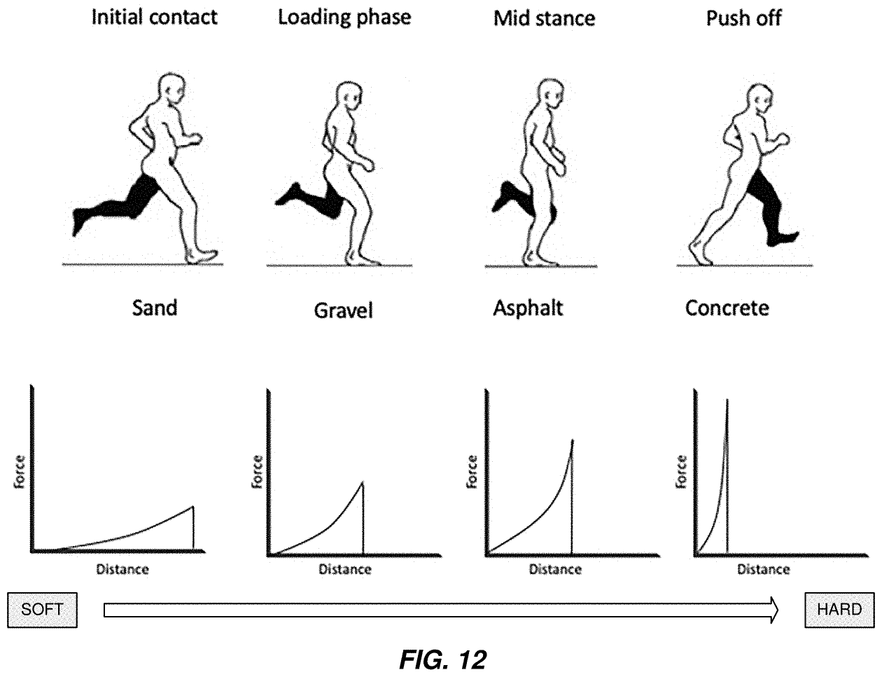

The Variable Mechanical Properties Shoe VMPS proposes construction of a running shoe that is highly compliant and flexible at initial impact, progressively becoming more and more non-compliant during (%) stance phase of running gait, and finally exhibiting a high level of non-compliance and stiffness at push off. VMPS proposes a principle that limits impact loads while still allowing more extensive utilization of our own tissues natural spring-like mechanisms. This novel concept has a dual benefit of preventing injury while at the same time maintaining running economy. FIG. 11 illustrates changing force-displacement curves over (% of stance phase) of VMPS.

The proposed concept is useful for both recreational and competitive runners, but especially important for the recreational runner whose lower extremities muscles, tendons, joints and ligaments) need exposure to natural mechanical stress and excursion, in order to maintain a certain level of health mitigating against deconditioning and contracture.

As an example, the inventor may have a mild chronic case of plantar fasciitis. He has no pain when he runs on sand. He has minimal discomfort when he runs on gravel and slightly more uncomfortable when he runs of asphalt. He has moderate pain when he runs on concrete.

He loses time, speed and energy efficiency when he runs on sand, however with no pain.

He gains time, speed and energy efficiency when he runs on cement, however with pain.

From direct experience he understands that sand is more compliant than gravel, which is more compliant than asphalt, which is more compliant than concrete.

Question: Can he have a shoe that acts like sand on initial contact, gravel at loading phase, asphalt at mid-stance and concrete at push off? This imaginary shoe is compliant and energy inefficient at initial contact and stiff and energy efficient at push off, with a steady graded variation of compliance and stiffness in the midsole based on the trajectory of point of force application during % stance phase. FIG. 12 illustrates graded variation in (shock absorption) during a stance phase of running gait cycle.

As noted, midsoles used in current cushioned shoes are generally uniform at nano, micro and mesoscale levels and by necessity have constant unchanging physical characteristics throughout the midsole.

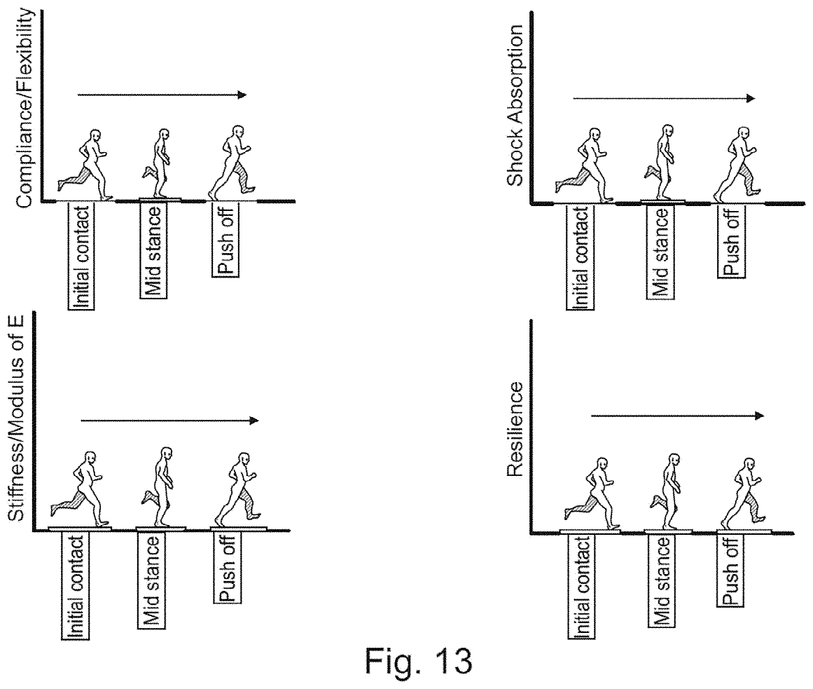

There is no structural characteristic that would produce “shifting” mechanical properties during the stance phase of gait. Stiffness (Modulus of Elasticity), shock absorption and resilience remain constant and unchanging as the VGRF traverses, for example from posterior to anterior, during the stance phase of the running gait cycle. FIG. 13 illustrates cushioned running shoe midsoles including compliance, stiffness, shock absorption, resilience that are all constant, and unchanging over time during the stance phase of the running gait cycle (% stance phase).

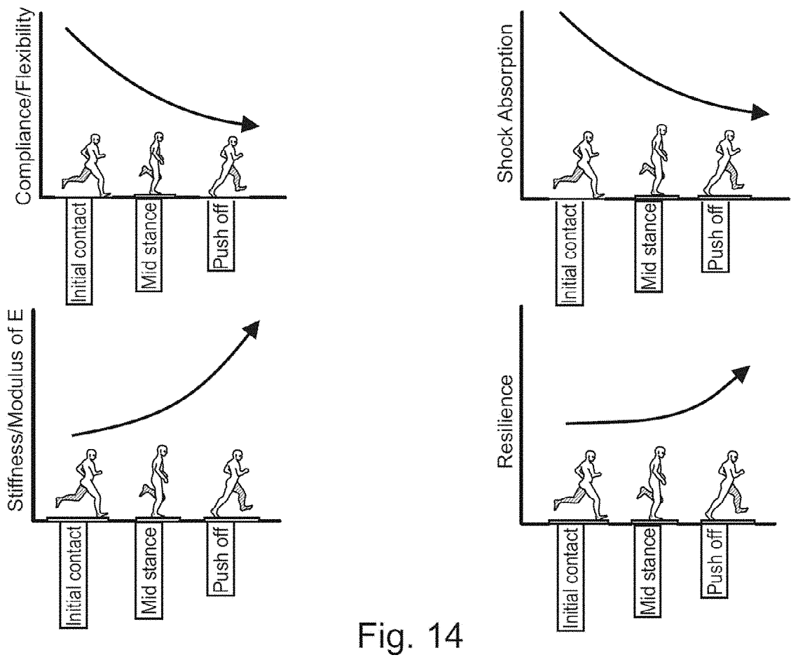

Conversely, VMPS has certain structural characteristics, at both microscopic and macroscopic levels, that effectuate a quantitative and qualitative change in the value of these variables during the stance phase of the running gait cycle. FIG. 14 illustrates a variable material properties shoe (VMPS) midsole including graded compliance, stiffness, shock absorption, resilience over (%) stance phase having varied, graded, non-homogenous structure of the “midsole” producing certain function during stance phase of gait.

VPMS therefore integrates the various qualities of the midsole (stiffness, compliance, shock absorption, resilience, flexibility) over time during the stance phase of the running gait cycle. In this manner the recreational runner has maximum shock absorption when most needed at initial impact, and maximum stiffness and resilience when most needed at push off. The runner does not have to settle for a “midway compromise” of all these variables combined into a single “average” value, throughout the midsole.

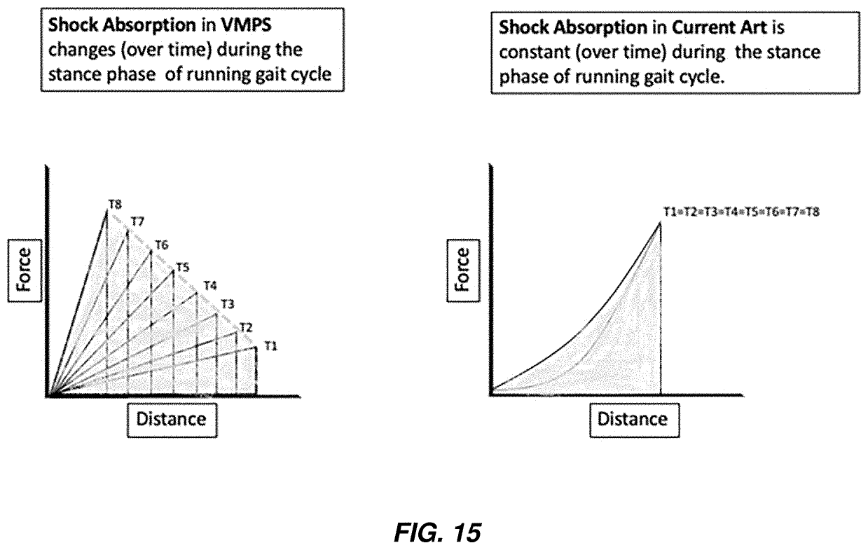

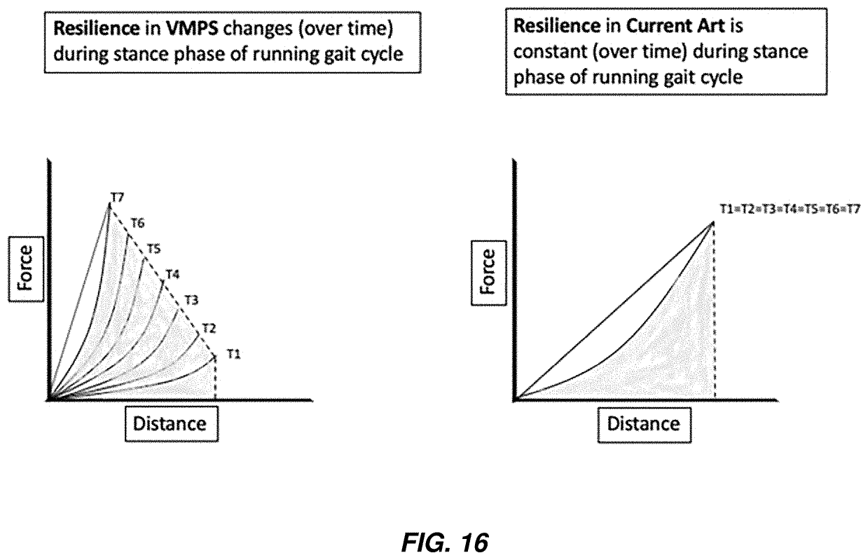

Finally, the concepts propounded above may prove to provide a higher quantity and quality of shock absorption and resilience for the competitive athlete who is concerned with attaining maximum shock absorption and resilience for long distance running, as seen by the graphs in FIG. 15 and FIG. 16. FIG. 15 illustrates a graded VMPS midsole having different quality and quantity of shock absorption as compared with highly cushioned shoe midsoles. FIG. 16 illustrates a graded VMPS midsole having different quality and quantity of resilience as compared with highly cushioned shoe midsoles.

In order to achieve these functionalities in the structure of the shoe we propose construction of a graded non-homogenous midsole that can be constructed (at the nanoscale, microscale, mesoscale, and macroscale levels). This special midsole is highly complaint and shock absorbing at initial impact, gradually and progressively becoming stiffer (non-compliant), eventually reaching maximum stiffness at push-off. The proposed graded non-homogeneous midsole of the VMPS midsole will likely exhibit some anisotropic characteristics.

Within the recreational runner population, the optimal shoe for each individual runner could be vastly different. A young fit runner with no gait abnormality and a low body mass index (BMI), requires a different shoe than an older runner with a high BMI, underlying knee arthritis, and an asymmetrical gait. The VMPS midsole can therefore be customized for each individual runner not only to allow locomotion without inducing disuse atrophy and injuries; but also, to allow normalization of asymmetrical gait, such that the presence of chronic weakness and contracture do not limit or discourage human locomotion. The VMPS midsole can also be customized for the individual runner to optimize performance. The capacity to move more freely without fear of pain, provides the capacity to remain metabolically and cardiovascularly healthy, which can have dramatic epidemiological consequences with decreases in incidence of diabetes, heart disease and osteoporosis.

In order to conceive of such a construct, we review the two phases of the gait cycle: the stance phase and the swing phase. The gait cycle begins when one foot makes contact with the ground and ends when the same foot makes contact with the ground again. The swing phase begins when the toe comes off the ground and ends just before the foot makes contact with the ground again. The stance phase begins when your foot makes contact with the ground and ends with toe off. Generally, it is divided into three stages, initial contact, midstance (including loading phase) and propulsion (toe off or push off).

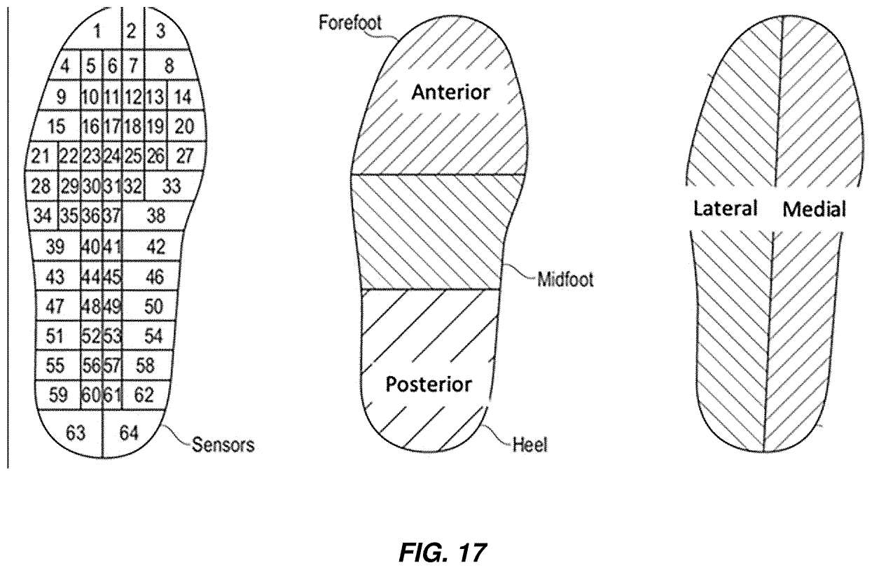

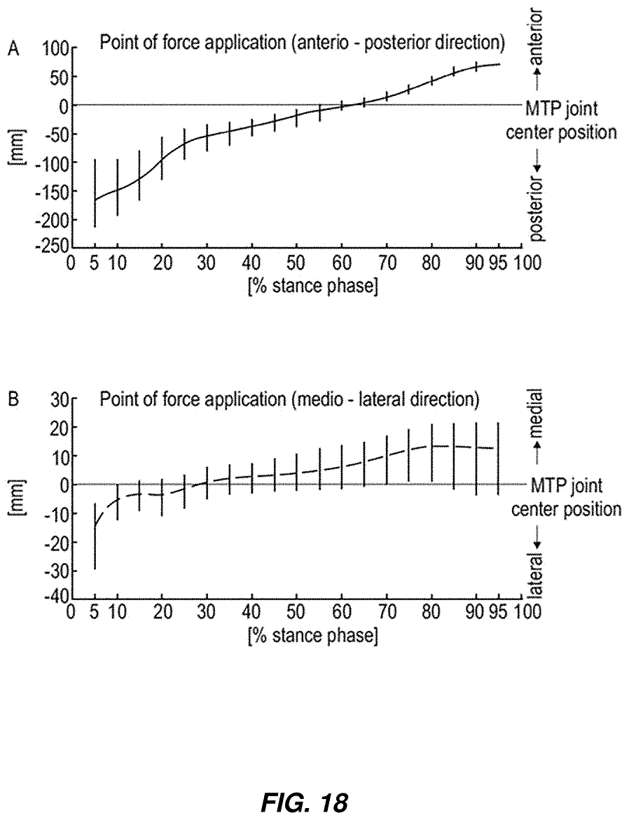

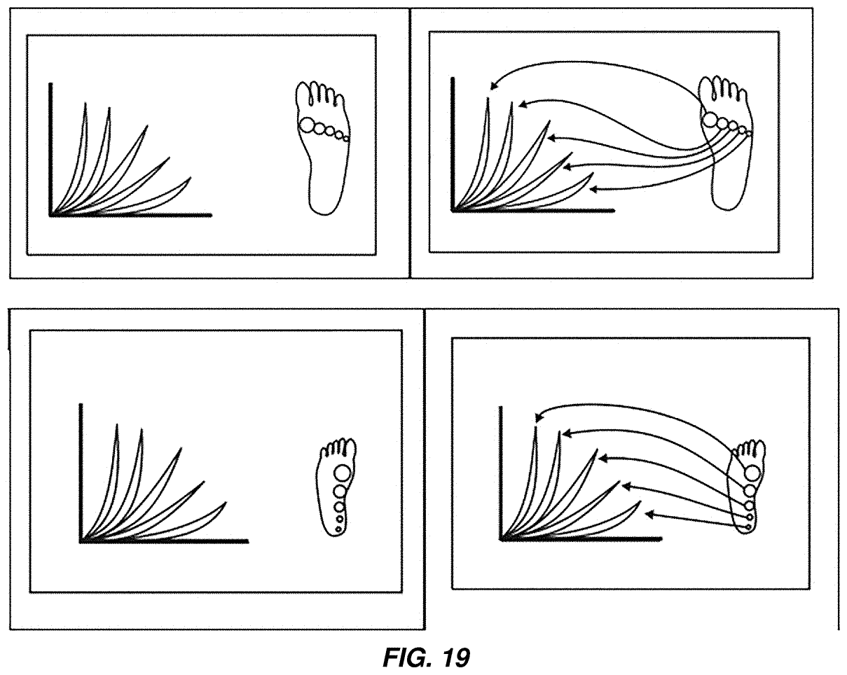

Studies have shown that if we measure the “trajectory” of how force is applied to the plantar aspect of the foot during the stance phase of gait, that a “point of force application” generally moves from posterior to anterior and/or from anterolateral to anteromedial during the stance phase of gait. FIG. 17 illustrates an arrangement of pressure sensors in mini regions of a segmented (tessellated) foot plate and a presentation of anterior, posterior, medial and lateral aspects of a foot plate. FIG. 18 illustrates a point of force application trajectory including a posterior anterior direction for a heel strike (rear foot) runner and lateral-medial direction for a midfoot strike (non-rear foot) runner.

This observation suggests that the structure of a VMPS midsole can be constructed in non-homogenous way to be complimentary to 1. VGRF and 2. Point of force application trajectory, during the % stance phase; such that the sole exhibits varying and graded (+/− monotonic) physical and mechanical properties. FIG. 19 illustrates a force versus displacement curves of VMPS midsoles showing varying graded stiffness from low to high based on % of stance phase, responsive to point of force trajectory including circles represent stiffness (larger circles representing increasing levels of stiffness; increasing levels of stiffness and associated (force-displacement curves) complementary to an anterolateral to anteromedial point of force application trajectory; and increasing levels of stiffness and associated (force-displacement curve) complementary to a posterior to anterior point of force application trajectory.

This midsole structure provides maximum energy absorption at initial impact and, based on the individual's force footprint, becomes progressively stiffer during progression of the stance phase. The VMPS concept allows multiple benefits including: 1. Protection against large impact loads at initial impact 2. Allowing progressive exposure of the muscles, tendons and ligaments to stress, engaging the natural spring-like mechanisms so as to prevent disuse atrophy and injury 3. Being mindful and maintaining energy efficiency.

The VMPS structure entails a graded and non-homogenous midsole with varying physical (and mechanical) propertied designed to correspond to the individual runner's specific VGRF and point of force application trajectory.

The proposed cushioned VMPS sole will work as an in-series spring, similar to current highly cushioned shoes, but will provide graded physical properties throughout discretized, segmented, tessellated portions of the midsole based-on time (% of stance phase).

VMPS is more flexible at initial contact and stiffer at push off. The transition of stiffness is progressive and occurs at both small and large scales, and maybe designed to be monotonic or non-monotonic within portions or entire body of the midsole.

For example, in a rearfoot (RF) runner, where the point of force application trajectory is from posterior to anterior, the sole of the VMPS shoe becomes progressively stiffer from posterior to anterior. Conversely, in a midfoot (NRF) runner, where the point of force application trajectory is from anterolateral to anteromedial, the midsole of the VMPS shoe becomes progressively stiffer from anterolateral to anteromedial.

The graded non-homogeneous structure of the VMPS is created to correspond directly to an individual's specific “footprint” which is a function of (point of force application trajectory and VGRF).

In summary, the VMPS midsole protects against large initial impact transients, while at the same time allowing a progressive and gradual transference of gravitational and kinetic energy to the muscles, tendons, ligaments and joints of the foot and ankle throughout the stance phase of the running gait cycle. The structure and derived function of VMPS sole allows: 1. Adequate shock absorption at impact to prevent traumatic injury, 2. Proper exposure of the muscles and tissues to stress to prevent atrophy, contracture and repetitive stress injury, 3. Adequate stiffness at push off to maintain running economy.

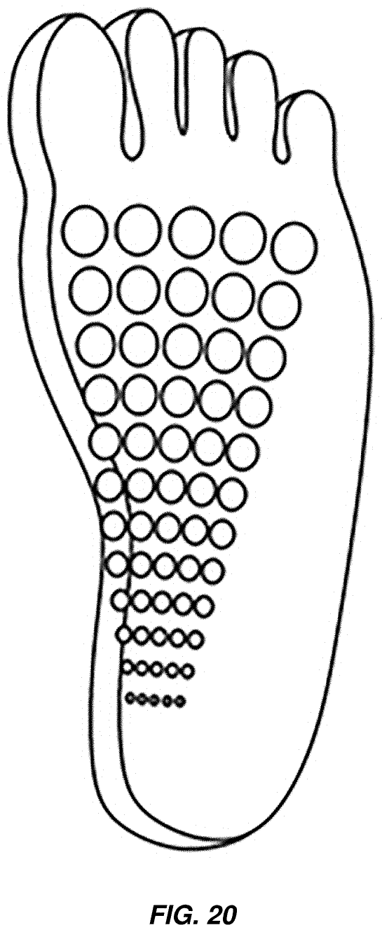

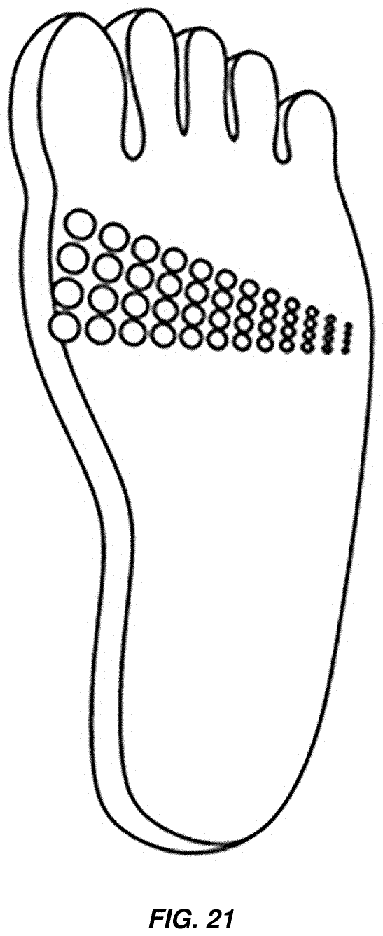

As an example, in a rearfoot (RF) runner, the point of force transition occurs from posterior heel to anterior midfoot or forefoot. The cushioned sole for this particular runner would provide stiffness properties that progressively (in a graded and non-homogenous fashion) increase from posterior to anterior, moving over the roll over axis of the foot during push off, in line with the first and second metatarsal phalangeal MTP joints. FIG. 20 illustrates a monotonic graded non-homogenous increase in stiffness in VMPS midsole from posterior to anterior with larger circles representing increasing levels of stiffness.

Conversely, in a midfoot (NRF) runner, the VMPS midsole would show stiffness properties that progressively increase (in a graded and non-homogenous fashion) from anterolateral to anteromedial toward the first and second metatarsal phalangeal joints. FIG. 21 illustrates a monotonic graded non-homogenous increase in stiffness in VMPS midsole from anterolateral to anteromedial with larger circles representing increasing levels of stiffness.

Additionally, many recreational runners have poor asymmetrical kinematic gait patterns due to old injuries. For example, a runner may have an old injury to her left knee, which over the years causes atrophy and scaring of the hamstring tendons leading shorter stride length on the left side.

Because of these types of phenomena, many of the recreational runners may exhibit some degree of asymmetry in their gait pattern.

A runner may exhibit a heel (RF) strike on the left foot and a midfoot (NRF) strike on the right foot and would therefore benefit from midsoles that have different graded stiffness profiles based on laterality.

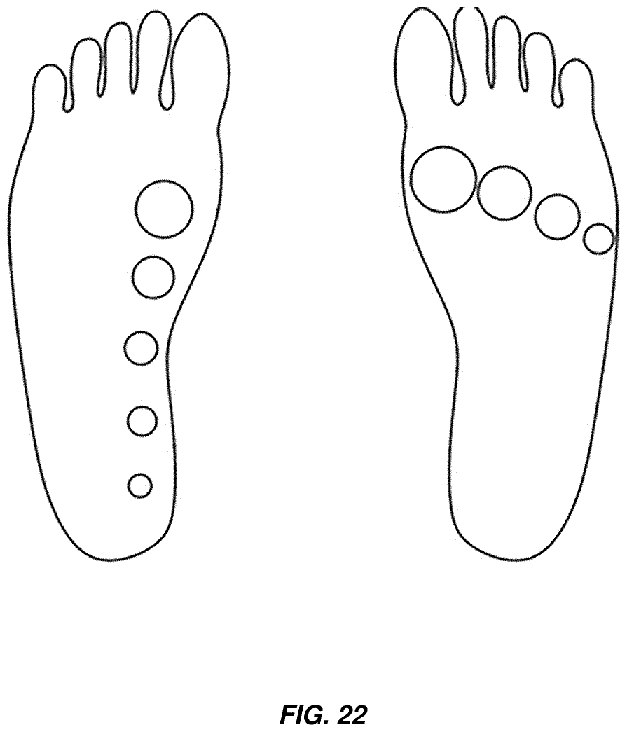

The optimal midsole for such a runner may include a left foot midsole with a stiffness profile that increases from posterior to anterior; and a right foot midsole with a stiffness profile that increases from anterolateral to anteromedial. VMPS therefore allows normalization of gait based on point of force application trajectory. FIG. 22 illustrates an asymmetrical gait with respect to point of force application trajectory with a left VMPS midsole becoming progressively stiffer from posterior to anterior, with a right VMPS midsole becoming progressively stiffer from anterolateral to anteromedial.

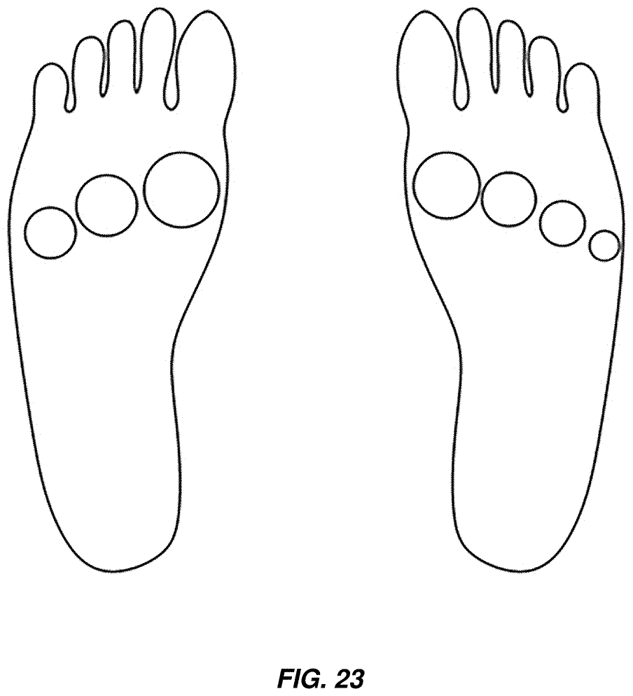

Conversely, a runner may have an asymmetric gait with respect to the magnitude VGRF, such that VGRF is higher on the right than on the left foot. The optimal midsole for this runner would require that the stiffness profile of the left midsole progress from moderate to high, while the stiffness profile of the right midsole progress from mild to moderate. VMPS therefore allows normalization of gait with respect to VGRF. FIG. 23 illustrates an asymmetrical gait with respect to VGRF with a stiffness profile of a left VMPS midsole progressing from moderate to high, with a stiffness profile of a right VMPS midsole progressing from mild to moderate.

Finally, not all runners exhibit the characteristically “typical” rearfoot or midfoot strike patterns described in the literature. Runners may exhibit atypical point of force application trajectories that do not conform with the standard well described strike patterns.

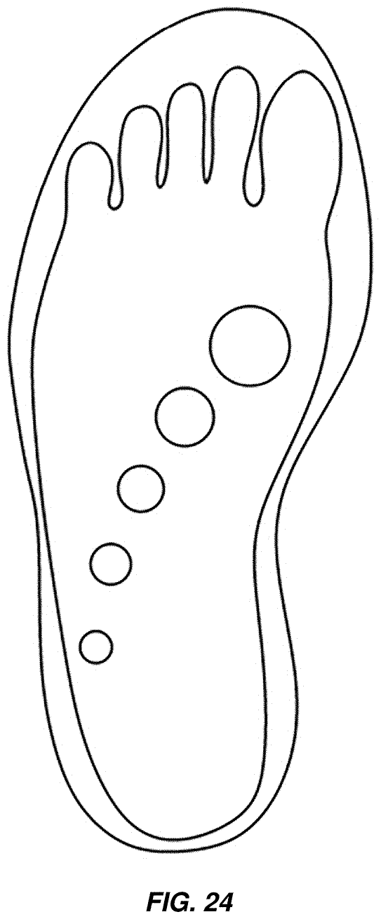

A “hybrid” strike pattern may, for example, may follow a “posterolateral to anteromedial” trajectory, as opposed to the standard posterior to anterior or anterolateral to anteromedial trajectories. FIG. 24 illustrates an atypical hybrid point of force application trajectory from posterolateral to anteromedial.

In order to construct a highly customized pair of VMPS soles, a runner's force footprint can be evaluated with force sensors placed in mini quadrants within a segmented/tessellated foot plate to assess the vertical ground reaction force VGRF and point of force application trajectory for each of the left and right feet.

A runner may have a rearfoot (RF) strike on the left side and a midfoot (NRF) strike on the right side. Many times, situations like this are based on poor mechanics related to old injuries. For example, with chronic left great toe injury, a runner may have a pronounced left foot heel strike with an VGRF of 900N; conversely, he may have a right midfoot strike with a VGRF of 500N. This runner's of point of force application trajectory for the left foot progresses from posterolateral to anteromedial; while his right foot point of force application trajectory progresses from anterolateral to anteromedial.

The running shoe customized for this runner would have left and right shoes with different physical properties corresponding to the individual's force footprint.

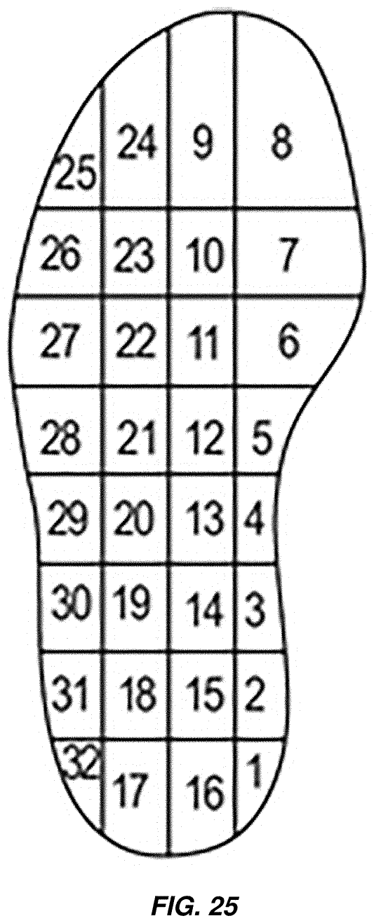

Considerations for construction of VMPS shoe for this hypothetical runner are illustrated in the drawings, particularly FIG. 25-FIG. 32.

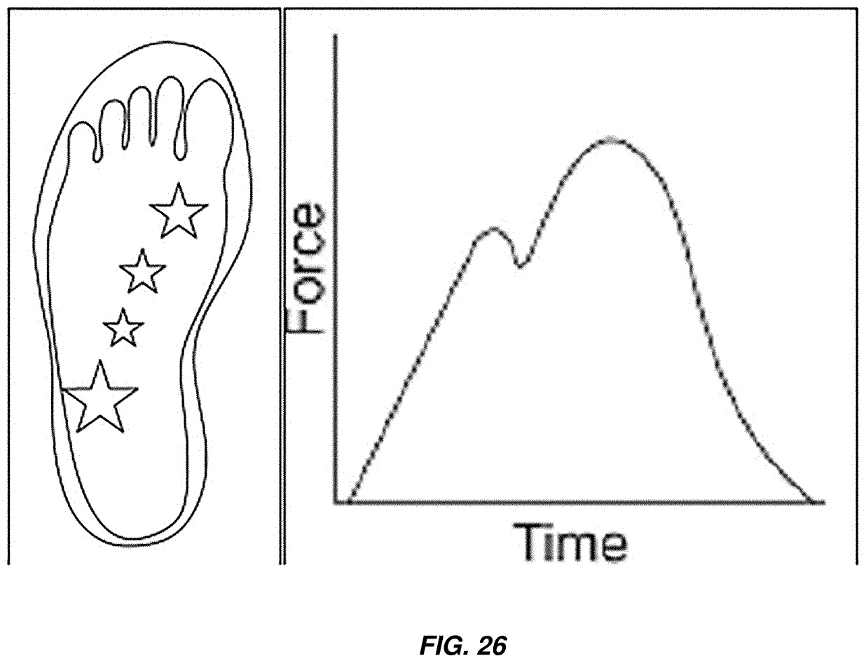

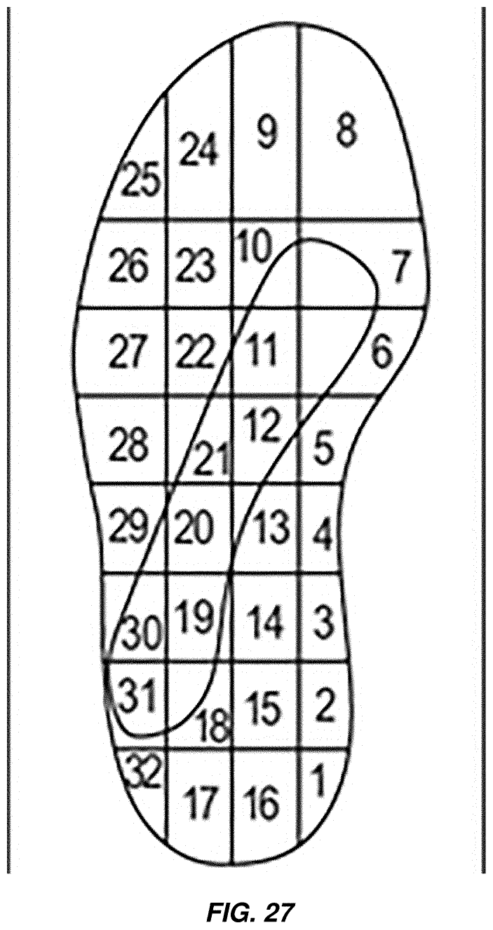

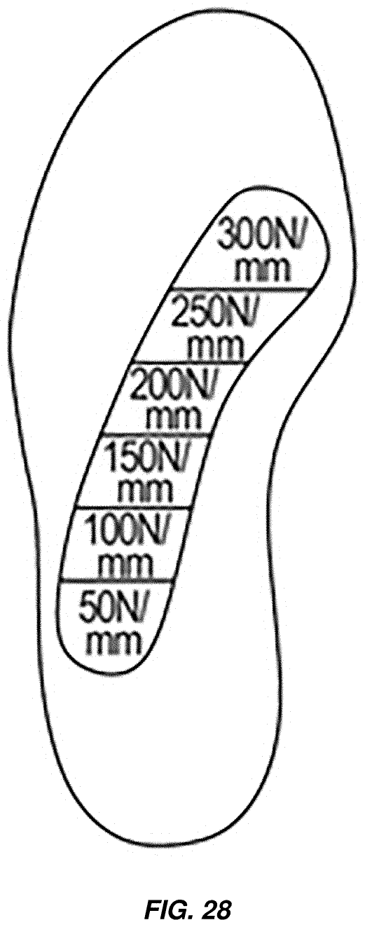

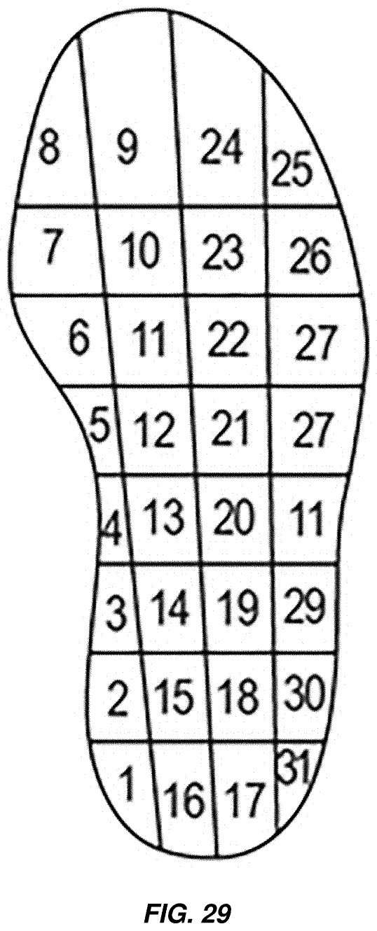

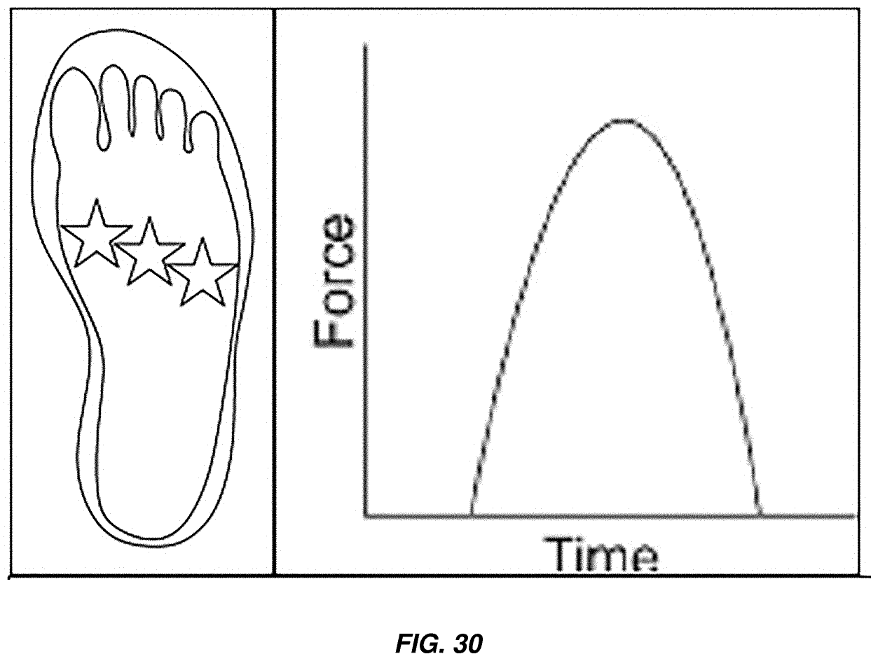

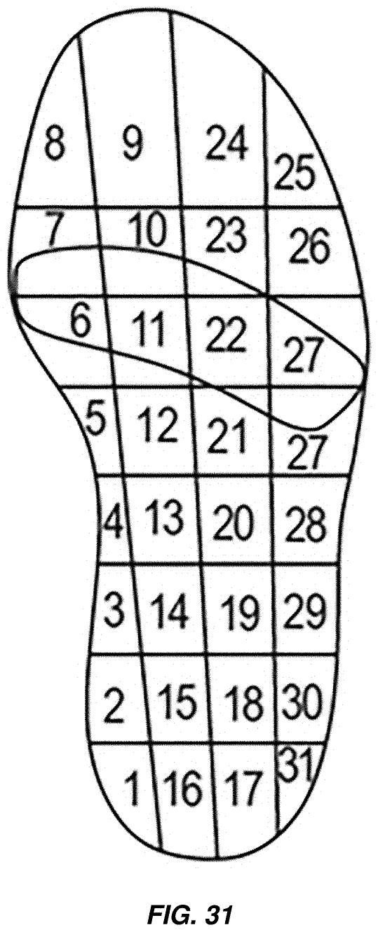

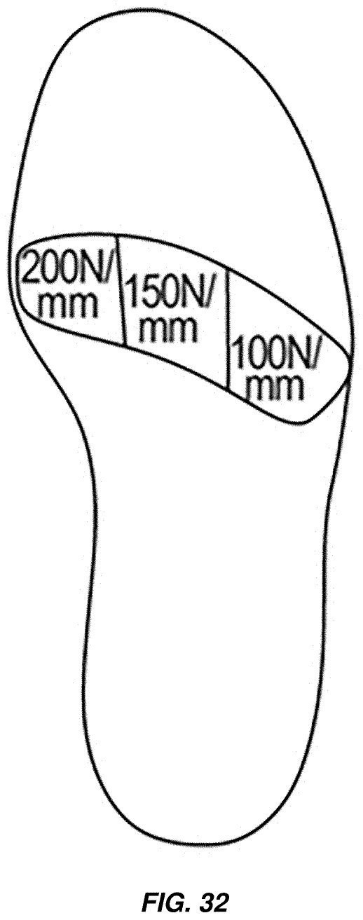

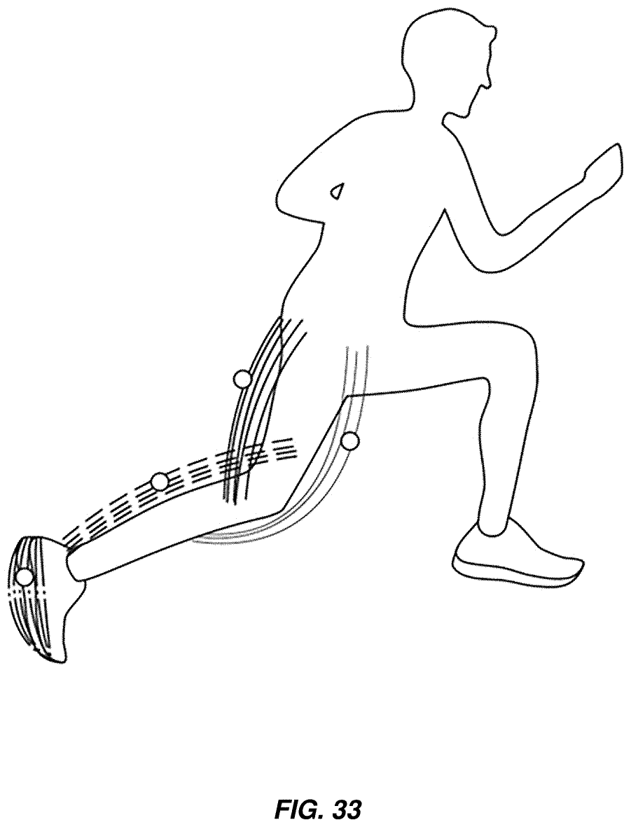

Evaluation of this runners force footprint may show, for example, that the left heel strike has a high VGRF that progresses from posterolateral quadrant (31) to anteromedial quadrant (7); which then necessitates construction of a midsole with a stiffness profile that spans from very soft to hard (i.e. from 40N/mm to 300N/mm). FIG. 25-FIG. 28 illustrate steps in creation of a left VMPS midsole for a runner with a high VGRF left heel strike pattern progressing from posterolateral to anteromedial. FIG. 25 illustrates segmentation of foot plate with mini quadrant force sensors. FIG. 26 illustrates a measurement of a “force footprint”=VGRF and point of force application trajectory. FIG. 27 illustrates a superimposition of a point of force application on a segmented foot plate; and FIG. 28 illustrates creation of a graded stiffness profile complementary to point of force trajectory. FIG. 29-FIG. 32 illustrate steps in creation of a moderate VGRF right midfoot strike pattern progressing from anterolateral to anteromedial, FIG. 29 illustrates segmentation of foot plate with mini quadrant force sensors, FIG. 30 illustrates a measurement of a “force foot print”=VGRF and point of force application trajectory, FIG. 31 illustrates a superimposition of a point of force application on a segmented foot plate; and FIG. 32 illustrates creation of a graded stiffness profile complementary to point of force trajectory.

Conversely, the force footprint evaluation may show a right midfoot strike, with a moderate VGRF that progresses from anterolateral quadrant (27) to anteromedial quadrant (6); which then necessitates construction of a midsole with a stiffness profile that gently spans from low intermediate to intermediate from anterolateral to anteromedial (i.e. from 100N/mm to 200N/mm).

Therefore, recreational runners will benefit enormously from custom VMPS shoes that absorb impact loads at the right time and right place for each of the runner's left and right feet, while at the same time not excessively inhibiting the runner's own natural inherent spring-like mechanisms, and therefore not inducing disuse atrophy, contracture and repetitive stress injury.

The VMPS sole can be created for any runner to maximize her ability to run or walk without experiencing pain and inflammation, while avoiding aggravation and worsening of pre-existing deficits, and maintaining a certain level of metabolic and cardiovascular conditioning, which may have dramatic individual health and socioeconomic consequences.

As a secondary consideration, the question arises as to whether the information gathered and used in the construction of the VMPS can additionally be used for diagnostic and therapeutic purposes.

Collection of large data sets of force footprints and associated abnormal gait kinematics provides the opportunity of multi-variate analysis. For example, a certain force footprint may be highly correlated with a certain asymmetric gait, which may occur as a result of a certain chronic injury. Therefore, an old left hamstring injury in soccer may result is a contracture of the muscle, which may result in a short stride length on the left side, which may result in a high VGRF heel strike of the left foot. Whereas on the right side the same runner may have no deficiencies (no old injury, normal hamstring, normal stride length, moderate VGRF midfoot strike). We term the phenomena of correlating force footprints with abnormal gait kinematics VMPS diagnostics.

It is also conceivable that enhanced diagnostics can be employed once the VMPS process is established. More granular and detailed data regarding a musculoskeletal deficiency may be collected with more direct (minimally invasive) sensing of affected tissues; which can then be used for highly specific targeted therapy program to rectify the tissue deficiencies and the pathologic gait.

It is noteworthy, that in U.S. patent application Ser. No. 16/375,736, hereby expressly incorporated by reference thereto in its entirety, we described implanting of small microchips to muscles, tendons, ligaments and bone, in order to provide Prophylactic Monitoring-Point of Care Testing in Orthopedics (PM-POCT). These biosensing transducers were integrated with wireless communication elements to transmit sensing signals to external receiving device. Incorporation of these types of microchips in lower extremity muscles involved in locomotion allow clinicians to access and correlate particular force footprints, levels of musculotendinous mobility and tissue metabolites with certain mechanical deficiencies, injury or disease. (i.e. contracture, inflammation, infection). See paragraphs [0293], [0294], [0295], [0345], [0349] and [0350] from the incorporated application Ser. No. 16/375,736.

Wireless biological electronic sensors have been created by integrating a bio-receptor sensing transducer with wireless antennas. The wireless aspect of (biological electronic systems) are classified into following categories: wireless radio frequency identification, wireless acoustic waved based biosensors, wireless magneto elastic biosensors, wireless self-powered biosensors and wireless potentiostat-based biosensors.

To develop wireless biological electronic sensors, a sensing transducer is immobilized (attached) to bioreceptor to make a biosensing transducer. This biosensing transducer is further integrated with a wireless communication element to transmit sensing signals to external receiving device.

Several types of sensing transducers have been used and include electrochemical electrodes, transistors, resistors, capacitors, surface acoustic wave electrodes, magnetic acoustic plates, magnetoelastic ribbons.

The ability to apply, through small incisions or percutaneously, small biosensors within tendons, bones, and ligaments provides the possibility of Prophylactic Monitoring Point of Care Testing in orthopedics (PM-POCT).

In the example of the heel striking runner discussed above, a biosensor applied to the calcaneus, tibia, plantar fascia, Achilleas tendon and the tarsometatarsal ligaments of the foot, with ability to measure force (loading), displacement (LVDT sensor), directionality (IMU inertial measuring units), and inflammatory metabolites (i.e., mast cells, macrophages, cytokines, chemokines, histamine, and the like) can not only detect whether microtears and inflammation are actually occurring through the (PM-POCT) process, but also determine WHY they are occurring.

In the example note above, the heel strike runner with very tight hamstring, adductor (groin muscle) and hip flexors (iliopsoas) will have a very short gait pattern (or stride length) without the ability to full flex and extend the hips producing less forward propulsion in the horizontal direction and more upward and downward motion leading to large vertical ground reaction forces GRF, and large vertical loading rate. This alteration in mechanics can clearly lead to a stress fracture of the calcaneus or tibia (and/or damage to the knee joints) for example. Similarly, any imbalance in the biomechanical function of the lower extremity musculotendinous system (typically tight and contracted muscle units) can lead to excessive loading (over repeated cycles) of certain bone and joints causing microtears, tendinitis, stress fractures and other repetitive stress injuries.

These specialized mechanical and/or biologic biosensors (bioreceptor and transducer) integrated with wireless communication have the capacity to measure and transmit data including force, acceleration, displacement, metabolic and inflammatory byproducts. They can be applied to any desired muscle group, tendon, ligament and fascial tissue (i.e. hamstrings, quadriceps, gastrocsoleious, plantar fascia and the Iliotibial band) involved in locomotion in order to determine atrophy, inactively, scarring, contracture, inflammation and disease of the tissues.

Therefore, asymmetric and pathologic gait can be initially evaluated non-invasively with VMPS diagnostics, and subsequently additional enhanced diagnostic information can be acquired from biosensors implanted in tissues.

This protocol can lead to better understanding of pathologic or painful gait, which can lead to development of an enhanced diagnostic (POC) criteria, which can lead to specific targeted therapy protocols for resolution of pathologic gait.

In other words, certain force footprints are associated with certain chronic musculoskeletal injuries, which result in abnormal gait kinematics. Once a causal relationship between force footprint, abnormal gait and chronic injury is established; enhanced diagnostics with implanted microchips can be deployed. This secondary diagnostic step can lead to better understanding of the pathophysiology, which leads to more effective treatment. FIG. 33 illustrates a specialized microchips embedded in major lower extremity muscle groups, including quadriceps, hamstrings, gastrocsoleious, and plantar fascia.

An example of the VMPS diagnostics guiding enhanced diagnostics and targeted therapy is as follows. A runner with an old injury to the right great toe metatarsal phalangeal joint may develop a high VGRF heel strike in order to protect the arthritic joint in the forefoot. A microchip implanted in the plantar fascia would recognize reduced tissue excursion (tensile load) and/or inflammatory metabolites in the plantar fascia and the windlass mechanism of the foot. A targeted therapy program would conceivably involve stretching, myofascial release and modalities such as ultrasound directly to the plantar fascia; along with mobilization of the right great toe metatarsal phalangeal joint. In this manner a specific diagnosis of plantar fascia contracture is obtained through a multi-scale (Point of Care) diagnostic system, which leads to a targeted therapy program that can realistically improve a runner's gait.

Furthermore, biofeedback methods could additionally be employed to correct and gain control of deficient gait kinematics.

This can be done for example through visual, audio and tactile biofeedback. Therefore, a runner can have real-time information about her bilateral foot strike patterns (point of force application trajectory and magnitude of VGRF) instantaneously available through multiple microchip sensors embedded within the segmented mini-quadrants of a VMPS midsole. Thus, for every step where a runner produces an excessively high VGRF and/or any atypical strike pattern; visual, auditory and tactile information can be immediately available and instantaneously feedback for correlation and corrective maneuvers.

This information is transferred wirelessly and made available to the runner: 1 visually through wearable computer glasses (smart glasses) equipped with infographics (graphic visual representation of information), 2. through tactile vibrations and haptic technology using wearables such as watches, earrings, necklaces and/or clothing, or 3. Through auditory feedback using earpiece technologies such as headphones, earbuds and other electroacoustic transducers, converting electrical signals into sound.

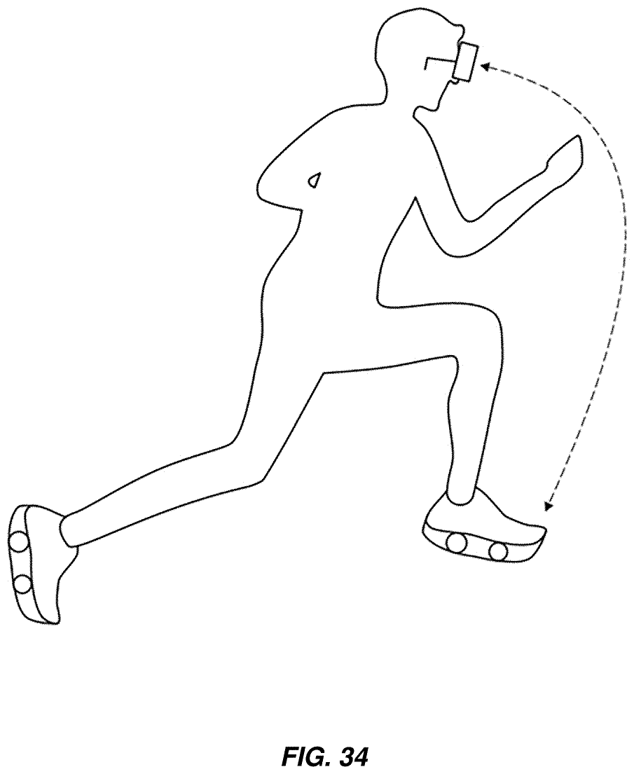

Hence, the runner can be instantaneously aware and conscious of her proper and/or poor gait kinematics and make immediate corrective adjustments. FIG. 34 illustrates a biofeedback modality including an instantaneous feedback regarding runner's VGRF and point of force application trajectory sensed by mini-quadrant force sensors in VMPS midsole and transmitted to runner's smart glasses wirelessly, and displayed graphically.

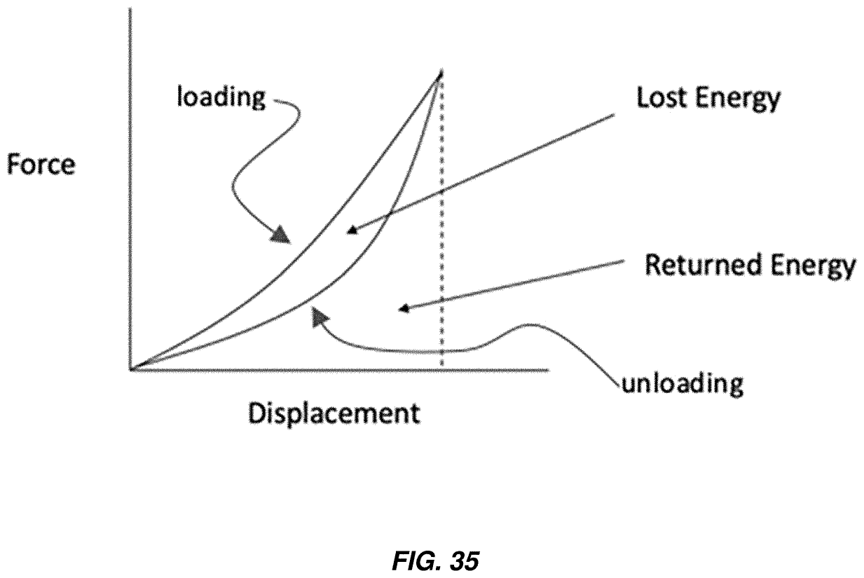

Virtually all modern running shoes have midsoles made from various foam materials that to varying degrees cushion impact, store and return mechanical energy. The amount of energy stored by a foam material depends on its compliance—the amount of compression that occurs when loaded with a certain force. Compliant foams are commonly described as soft; however, all foam is viscoelastic in that they dissipate energy as heat. The percent of stored mechanical energy that is returned is called resilience. FIG. 35 illustrates a force versus displacement curve for typical midsole foams used in running shoes, showing loading and unloading of the foam, with energy lost as hysteresis, energy returned as resilience, maximum energy absorbed by the shoe as a sum of lost energy and returned energy.

Some material/surfaces are compliant but have low resilience and thus increase the energic cost of running.

Shoe manufacturers have continued to pursue foams and constructs that have a perfect combination of compliance and resilience to provide comfort and reduce the energetic cost of running.

As we have discussed earlier, this approach may decondition the recreational runner by hindering the natural spring-like functioning of the foot, which ultimately leads to injury.

Running shoe midsole materials may include polymers such as polyvinyl acetate (PVA), polyurethane (PU), ethylene vinyl acetate (EVA), Polyether block amide (PEBA), isoprene, neoprene, or a combination of these types of polymers. Specific polymers are injected with gas to form foam. A given formulations of foams and polymers are proprietary data and are configured to provide specific mechanical properties to the midsole of the shoe.

A polymer is a large molecule or macromolecule composed of many repeated subunits. Polymers range from familiar synthetic plastics such as polystyrene to natural biopolymers such as DNA and proteins that are fundamental to biological structure and function. Polymers both synthetic and natural are created via polymerization of many small molecules known as monomers.

Their consequently large molecular mass, relative to small molecule compounds produces unique physical and mechanical properties including strength, toughness, viscoelasticity. Polymers can be produced by linkage of repeating units with covalent bonds as well as non-covalent bonds. Natural polymers such as hemp, shellac, amber, wool, silk, and natural rubber have been used for centuries.

Synthetic polymers include polyethylene, polypropylene, polystyrene, polyvinyl chloride, synthetic rubber, phenol formaldehyde resin, neoprene, nylon, polyacrylonitrile, PVB, silicone and many more.

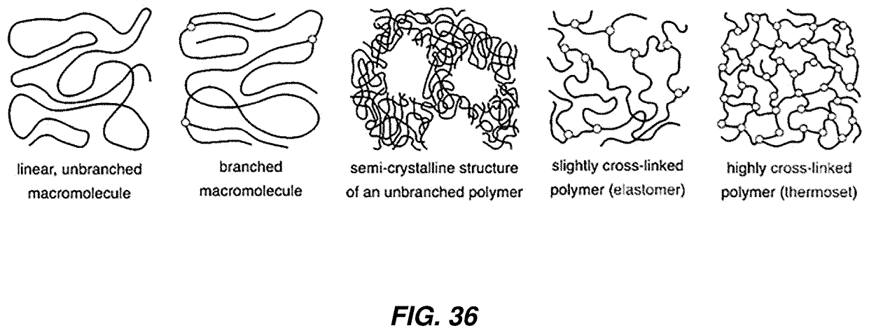

Polymerization is the process of combining monomers into covalently bonded chain or network. Polymers can be modified by processes such as oxidation, cross-linking, and endcapping. FIG. 36 illustrates polymer modification including branching, crosslinking, and crystals.

The basic property of the polymer is the identity of its constituent monomers. A second property of the polymer is its microstructure which relates to the arrangement of the monomers within the polymer at the scale of a single chain. Polymer nomenclature is generally based upon the type of monomer residues comprising the polymer. A polymer which contains only a single type of repeat unit is known as a homopolymer, while a polymer containing two or more types of repeat unit is known as a copolymer.

The microstructure of the polymer or configuration relates to the physical arrangement of the monomer residue along the backbone of the chain and can be modified to alter the physical and mechanical properties of the polymer. Examples of different microstructures include linear unbranched macromolecule, branched macromolecule, slightly cross-linked polymer (elastomer), and highly cross-linked polymer (thermoset). Similarly the architecture and shape, length of chain, monomer arrangement, stereochemistry (Tacticity), morphology (microscale ordering of polymer chains), cross-linking, crystallinity can all affect and modify the mechanical and bulk properties of a polymer (i.e. how the polymer behaves at the macroscopic scale), which includes but not limited to properties such as tensile and compressive strength, young's modulus, poisson's ratio, and toughness.

As an example, thermoplastic polyurethane (TPU) is a class of polyurethane plastics referred to as thermoplastic elastomers consisting of linear segmented block polymers composed of hard and soft segments. This polymers can be fine-tuned by chemists to produce varying and different mechanical properties. For example, in TPU, a greater ratio of hard to soft segments will result in a more rigid TPU, while the reverse is also true.

Additionally, polymer mechanical properties can be modified through copolymerization through addition of non-reactive side chains to monomers before polymerization. Additionally, properties such as strength and brittleness can be modified through addition of plasticizers (additives that alter the plasticity, viscosity, flexibility and toughness of a material).

Solid foams can be closed cell or open cell and in many cases is considered a multi-scaled system; forming bubbles at larger scale, interconnected films called lamellae at a smaller scale, and a liquid-air interface at the surface of the film at even a smaller scale. Open cell foams are generally softer. Closed cell foams have higher compressive strength. Generally, stiffness and mechanical properties of foams can be altered in a variety of ways including modification from open cell to closed cell, altering the geometry of the foam cells which results in different strength, stiffness and toughness per unit weight. Additionally, a special class of foams known as syntactic foam, contains hollow particles embedded in the material matrix. These spheres can be made of several materials including glass, ceramic, and polymers. Techniques such as these, generally allow modification of the mechanical properties of foam at microscale and macroscale levels.

In one embodiment of VMPS, the polymer foam for the midsole will be fabricated in such a way to produce graded non-homogeneous physical characteristics, such as stiffness and toughness; which will be designed to be complimentary to runners' particular force footprint (magnitude of VGRF and point of force application trajectory).

In U.S. patent application Ser. No. 16/693,214, hereby expressly incorporated by reference thereto in its entirety, we proposed the construction of a composite implant at the nanoscale, microscale and mesoscale levels where the matrix base metal is reinforced with a framework (or reinforcements) such as fiber, glass, polymers and other metals to produce a more sophisticated implant that is less dense, has anisotropic and viscoelastic properties and more closely replicates the structure of human bone, in order to allow smoother transition of force across the implant/bone interface, as well as more stable fixation of implant to bone.

The benefits of the composite implant include enhanced longevity of the implant, decreased the risk of fracture at the cellular level, better osteointegration, and avoidance of stress shielding and bone resorption.

In the current application, based on the same philosophy discussed in U.S. Pat. No. 10,299,930 and U.S. patent application Ser. No. 16/693,214, hereby expressly incorporated by reference thereto in their entireties, a variety of fiber strands or particles can be used as a framework (reinforcement) within the chosen polymer or polymer foam (matrix) to form “composite foams” with graded non-homogeneous physical characteristics (i.e. stiffness). The reinforcements may include various materials such as glass fibers, carbon fibers, cellulose, silicone and other high strength polymers such as aramid, to enhance structural stability and modify mechanical properties of the midsole polymer or polymer foam. These reinforcements can be provided in various forms such as particles, random chopped strands, unidirectional longitudinal or horizontal strands, laminates, and a variety of frameworks a graded and non-homogeneous polymer or polymer foam.

Furthermore, the mechanical properties of polymers can be altered at the nanoscale, microscale, mesoscale and macroscale levels, through changes in the architecture and shape of the polymer, for example by adjusting length of chain, monomer arrangement, stereochemistry (Tacticity), morphology (microscale ordering of polymer chains), cross-linking and crystallinity. In one embodiment of the VMPS sole, the alterations of the mechanical properties of the polymer can occur monotonically to provide a graded and non-homogeneous stiffness profile.

Similarly, alterations in stiffness profile of the midsole can also be achieved at the stage in which polymer foam is produced. One method of varying the stiffness profile of foam is to fabricate the foam such that there is a graded non-homogeneous transition of polymer foam from open cells to closed cells. For example, in one embodiment, the VMPS polymer foam is constructed such that the proportion of open to closed cells monotonically decrease/increase from one segment to another. Additionally, the architecture of the foam cells can be adjusted to provide a smooth transition of the stiffness profile from flexible to stiff at the microscale level. Similarly, plasticizers can be used to vary the degrees of stiffness and mechanical properties of foam cells.

These types of adjustments to the polymers, polymer foam, and polymer foam composites can provide the proposed variations in stiffness, compliance, shock absorption and resilience profile of the VPMPS midsole, in a progressive, graded, non-homogenous, (+/− monotonic) fashion.

Therefore, the VMPS sole may be fabricated through subtractive and/or additive manufacturing techniques in order to provide a graded variation in its mechanical and physical properties, at the micro and macroscale levels, which include but is not limited to properties such as tensile and compressive strength, young's modulus, stiffness, poisson's ratio, toughness and compliance.

The primary benefit of VMPS is to provide shoes that allow runners to utilize their own body's natural elastic spring-like mechanisms more effectively while providing shock absorption to protect against transient loads. The most important contribution of the VMPS concept is that runner's tissues will not atrophy and contract becoming prone to repetitive stress injury. Comfort and cushioning are maintained without having to pay the high price of disuse atrophy and deconditioning associated with highly cushioned shoes. VMPS integrates shock absorption and running economy over time during the (%) stance phase, rather than providing a uniform single best average of the two variables throughout the midsole.

The secondary benefit of VMPS is the capacity to deal with asymmetric and pathologic gait. Runners and walkers around the world will have the opportunity, through “laterality” customized VMPS, to have normalized and symmetrical gait mechanics with respect to VGRF and point of force trajectory. This concept provides the sensation of a gait closer to what they experienced in childhood before accumulating unilateral injuries. Old injuries lead to asymmetric gait which leads to uneven loading and unloading of the extremities, which leads to injury and pain; VMPS midsole can correct and normalize this deficiency.

The tertiary benefit of VMPS is development of newfound VMPS diagnostic capabilities. Analytical evaluation of force footprint data with variety of gait abnormalities allows for better understanding (cause and effect) of repetitive stress injuries associated with walking and running. Categorization and correlation of atypical force footprints with altered gait patterns allows for targeted therapeutics. A contracted hamstring muscle confirmed with VMPS diagnostics leads to a focused therapy regimen including mobilization, stretching, and modalities of the hamstring muscles, instead of generic stretching and strengthening.

Enhanced (POC) diagnostic capabilities can be achieved directly through embedded tissue microchips. Once a particular force footprint is associated with a particular gait abnormality and specific tissue contracture, the tissue in question can be further studied with microchips implanted through minimally invasive procedures. Additional data such as force and displacement measurements, as well as inflammatory markers can be measured. This additional diagnostic capability can then lead to 1. New classification of running injuries, and 2. Specific and targeted treatment protocols including therapy and/or surgery.

Additional therapeutic capabilities can be achieved through various biofeedback mechanisms, which can be employed to help the runner evaluate and correct pathologic gait patterns in a real-time setting.

Furthermore, this field is ripe for data collection and analysis with and incorporation of cognitive technologies. Multi-variate analysis of force footprints, microchip tissue data and various gait patterns will allow better understanding of pathophysiology of abnormal gait, which will ultimately lead to development of better products.

The adoption of the VMPS midsole reduces of running injuries and normalizes gait, as well, it can have major health and financial benefits for the individual, society, insurance companies and governments. Increased (painless) physical activity and movement, leads to better musculoskeletal, metabolic and cardiovascular health.

From the military perspective, many young recruits are physically, metabolically and cardiovascularly out of shape. During basic training, many sustain injuries such as stress fractures and tendinitis to their lower extremities. Shoe prescriptions, orthotics and highly cushioned shoes have not shown a benefit in decreasing the incidence of these disabling injuries in this population. The VMPS midsole concept allows rapid construction of custom footwear for recruits, so that they can immediately walk, march and run without sustaining disabling injuries and time off work.

Ultimately adoption of this concept can save billions of dollars for the government, military, corporations and society in general.

A quick summary:

1. Consider the words “soft-hard” in succession as you would in normal speech. That takes one second.

2. The stance phase of running gait is 0.3 seconds, that is (⅓) as long it takes for you to say “soft-hard”.

3. During the stance phase of your gait, in that 0.3 seconds, the VMPS midsole will provide a monotonically increasing stiffness (from soft to hard).

4. Depending on your foot strike, the progression will either be from posterior to anterior or from anterolateral to anteromedial.

5. You will have good shock absorption when you need it at initial impact (0.1 second of stance phase)

6. You will have good running economy at push off when you need it (0.3 second of stance phase)

7. Your tissues will get good exercise and remain healthy, so they won't atrophy and decondition leading to strain, tendinitis and stress fractures.

8. If you have asymmetric painful gait, VMPS provides a solution by normalizing your gait.

The system and methods above have been described in general terms as an aid to understanding details of preferred embodiments of the present invention. In the description herein, numerous specific details are provided, such as examples of components and/or methods, to provide a thorough understanding of embodiments of the present invention. Some features and benefits of the present invention are realized in such modes and are not required in every case. One skilled in the relevant art will recognize, however, that an embodiment of the invention can be practiced without one or more of the specific details, or with other apparatus, systems, assemblies, methods, components, materials, parts, and/or the like. In other instances, well-known structures, materials, or operations are not specifically shown or described in detail to avoid obscuring aspects of embodiments of the present invention.

Reference throughout this specification to “one embodiment”, “an embodiment”, or “a specific embodiment” means that a particular feature, structure, or characteristic described in connection with the embodiment is included in at least one embodiment of the present invention and not necessarily in all embodiments. Thus, respective appearances of the phrases “in one embodiment”, “in an embodiment”, or “in a specific embodiment” in various places throughout this specification are not necessarily referring to the same embodiment. Furthermore, the particular features, structures, or characteristics of any specific embodiment of the present invention may be combined in any suitable manner with one or more other embodiments. It is to be understood that other variations and modifications of the embodiments of the present invention described and illustrated herein are possible in light of the teachings herein and are to be considered as part of the spirit and scope of the present invention.

It will also be appreciated that one or more of the elements depicted in the drawings/figures can also be implemented in a more separated or integrated manner, or even removed or rendered as inoperable in certain cases, as is useful in accordance with a particular application.

Additionally, any signal arrows in the drawings/Figures should be considered only as exemplary, and not limiting, unless otherwise specifically noted. Combinations of components or steps will also be considered as being noted, where terminology is foreseen as rendering the ability to separate or combine is unclear.

The foregoing description of illustrated embodiments of the present invention, including what is described in the Abstract, is not intended to be exhaustive or to limit the invention to the precise forms disclosed herein. While specific embodiments of, and examples for, the invention are described herein for illustrative purposes only, various equivalent modifications are possible within the spirit and scope of the present invention, as those skilled in the relevant art will recognize and appreciate. As indicated, these modifications may be made to the present invention in light of the foregoing description of illustrated embodiments of the present invention and are to be included within the spirit and scope of the present invention.

Thus, while the present invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the present invention. It is intended that the invention not be limited to the particular terms used in following claims and/or to the particular embodiment disclosed as the best mode contemplated for carrying out this invention, but that the invention will include any and all embodiments and equivalents falling within the scope of the appended claims. Thus, the scope of the invention is to be determined solely by the appended claims.

What is claimed as new and desired to be protected by Letters Patent of the United States is:

1. A foot covering for a human foot, comprising:
a sole; and
an upper configured to couple said sole to the human foot; and
a variable materials properties structure incorporated into said sole.

2. The foot covering of claim 1 wherein a person wearing the foot covering on the human foot during a movement over a surface includes a gait pattern and wherein said variable materials properties structure includes a gait adapting arrangement responsive to said gait pattern.

3. The foot covering of claim 2 wherein said gait pattern includes a plurality of gait phases and wherein said gait adapting arrangement includes a first set of materials properties for said variable materials properties structure during a first gait phase and further includes a second set of materials properties for said variable materials properties structure during a second gait phase, said first gait phase different from said second gait phase and said first set of materials properties different from said second set of materials properties.

4. The foot covering of claim 3 wherein said sole includes an outsole and a inner sole disposed between said outsole and the human foot, said variable materials properties structure disposed in said inner sole.

5. The foot covering of claim 1 wherein said variable materials properties structure includes a distributed nonhomogeneous properties gradient over an expanse of said variable materials properties structure.

6. The foot covering of claim 5 wherein said distributed nonhomogeneous properties gradient is configured to correspond to an individual's specific footprint which is a function of a point of force application trajectory and a vertical ground reaction force.

7. The foot covering of claim 2 wherein said variable materials properties structure includes a set of gait-characterizing sensors responsive to said gait pattern during said movement over said surface.

8. The foot covering of claim 7 wherein said gait pattern results from a biomechanical condition for the human, said biomechanical condition including a degradation of said gait pattern with respect to a standard gait and wherein said set of gait-characterizing sensors are configured for an evaluation of said biomechanical condition during said movement.

9. The foot covering of claim 8 wherein said evaluation is configured to define a second variable materials properties structure configured to reduce said degradation.

10. A pair of foot coverings for a pair of human feet, comprising:
a pair of soles, one sole for each foot of the pair of feet; and
a pair uppers, each configured to uniquely couple one of said soles to one of the foots; and
a first variable materials properties structure incorporated into a first one of said soles; and
a second variable materials properties structure incorporated into a second one of said soles, said variable materials properties structures providing different variable materials properties.

11. A method for ameliorating a set of negative gait components of a gait pattern of a person moving over a surface while wearing a set of foot coverings, comprising:
incorporating at least one variable materials properties structure into a sole of at least one of the foot coverings of the set of foot coverings; and
compensating one or more of the gait components of the set negative gait components using said variable materials properties structure.

12. The method of claim 11 wherein said compensating step includes measuring elements of the set of negative gait components during movement contributing to a degraded gait, and customizing said variable materials properties structure to decrease biomechanical contributions to said degraded gait.

13. A method for evaluating a gait pattern of a person moving over a surface while wearing a set of foot coverings, comprising:
incorporating at least one variable materials properties structure into a sole of at least one of the foot coverings of the set of foot coverings; and
characterizing a set of gait elements of the gait pattern using said variable materials properties structure.
